SEQUENCE LISTING (12 sequences)

SEQ ID NO: 1 - protein, 10 aa, Artificial Sequence
  Gantenerumab VH CDR1
GFTFSSYAMS

SEQ ID NO: 2 - protein, 17 aa, Artificial Sequence
  Gantenerumab VH CDR2
AINASGTRTYYADSVKG

SEQ ID NO: 3 - protein, 17 aa, Artificial Sequence
  Gantenerumab VH CDR3
GKGNTHKPYGYVRYFDV

SEQ ID NO: 4 - protein, 12 aa, Artificial Sequence
  Gantenerumab VL CDR1
RASQSVSSSYLA

SEQ ID NO: 5 - protein, 7 aa, Artificial Sequence
  Gantenerumab VL CDR2
GASSRAT

SEQ ID NO: 6 - protein, 8 aa, Artificial Sequence
  Gantenerumab VL CDR3
LQIYNMPI

SEQ ID NO: 7 - protein, 126 aa, Artificial Sequence [sequence omitted]

SEQ ID NO: 8 - protein, 110 aa, Artificial Sequence
  Gantenerumab VL domain
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 9 - protein, 456 aa, Artificial Sequence
  Gantenerumab Heavy Chain
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  Gantenerumab Light Chain
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 231 aa, Artificial Sequence
  heavy chain Fab fragment for antibody binding to human transferrin receptor
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCCYLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 215 aa, Artificial Sequence
  light chain for antibody binding to human transferrin receptor
AIQLTQSPSSLSASVGDRVTITCRASGSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2024/0002483 A1
(43) Pub. Date: Jan. 4, 2024

(54) FAB HIGH MANNOSE GLYCOFORMS

(71) Applicant: Hoffmann-La Roche Inc., Little Falls, NJ (US)

(72) Inventors: Klaus JOERIS, Penzberg (DE); Neslihan OEZDEN, Penzberg (DE); Wolfgang RICHTER, Basel (CH); Britta SCHMIDT, Penzberg (DE); Carsten HOFMANN, Basel (CH); Wilma LAU, Penzberg (DE); Roland STAACK, Penzberg (DE)

(21) Appl. No.: 18/037,071

(22) PCT Filed: Nov. 4, 2021
(86) PCT No.: PCT/EP2021/080692

§ 371 Date: May 15, 2023

(30) Foreign Application Priority Data
Nov. 16, 2020 (EP) 20207804.4

(51) Int. Cl. C07K 16/18; C07K 16/28

(52) U.S. Cl. CPC C07K 16/18; C07K 16/2881; C07K 2317/41; C07K 2317/55; C07K 2317/31; A61K 2039/505

(57) ABSTRACT

The present invention relates to glycosylation patterns at the Fab portion of a monoclonal antibody and methods for the regulation during culture of a microorganism expressing a monoclonal antibody with regulated content of high mannose Fab glycoforms.

### FIELD OF THE INVENTION

The present invention relates to glycosylation patterns at the Fab portion of a monoclonal antibody and methods for the regulation during culture of a microorganism expressing a monoclonal antibody with regulated content of high mannose Fab glycoforms.

### BACKGROUND

The key therapeutic or diagnostic properties of monoclonal antibodies are strongly related to the post-translational process of glycosylation. The glycan confirmations are important because they can influence secretion, solubility, receptor recognition, antigenicity, bioactivity and pharmacokinetics. IgG antibodies are typically glycosylated on the Fc region, although about 20% also contain non-conserved N-glycosylation sites in the variable region (Zhang et al., Drug Discovery Today 21 (2016) 740-765).

The N-linked glycosylation of the Fc region of IgG has important structural functions, including enhancing stability and influencing the folding of the Fc part (Higel et al., European Journal of Pharmaceutics and Biopharmaceutics 100 (2016) 94-100). Fc-linked glycans influence the effector function of the antibody by altering the three-dimensional structure of the protein, and thereby the binding to Fc-γ-receptors.

The role of glycosylation in the variable region is not, however, so clear. Zhang et al., (supra) propose that most V-region glycosylation sites are located on solvent-exposed loops and are readily accessible to endogenous lectins. Although typically containing fully processed, sialylated glycans, with a high incidence of bisecting GlcNAc residues, some oligomannose glycans have also been found in the CDR antigen-binding regions. It has been postulated that these oligomannose glycans contribute to pathological processes by binding to lectin domains of the innate immune system, blocking their signalling functions.

Typically the glycans of the Fab have been described as biantennary complex-type structures that are galactosylated and, in contrast to Fc glycans, highly sialylated. The function of these oligosaccharides has not been fully elucidated but there are suggestions that glycosylation of the variable regions can have positive, neutral or negative influences on antigen binding (Biermann et al., Lupus 25 (2016) 934-942 and Jefferis, Nature 8 (2009) 226-234). Similarly, various studies have been performed to investigate the relationship between N-glycosylation and pharmacokinetics (PK), but the findings are largely contradictory. Millward et al., Biologicals 36 (2008) 41-47 discovered that there is no significant difference in the clearance rates of antibodies glycosylated in the variable region from those with no glycosylation in the variable region, or between antibody enriched for complex type or enriched for high mannose type oligosaccharides at the Fc glycosylation site, thus suggesting that glycosylation has little or no impact on the pharmacokinetic behaviour of a monoclonal IgG1 antibody. Finke & Banks., UW Tacoma Digital Commons, SIAS Faculty Digital Publications (https://digitalcommons.tacoma.uw.edu/ias_pub/825) note that the biological role and therapeutic consequences of Fab glycosylation is much more obtuse than the relatively well characterised structure-function relationship of Fc glycans on IgG effector function. They conclude from an assessment of a number of studies that the effect of a given Fab glycan on biological properties and therapeutic metrics of an IgG drug is difficult to predict. Van de Bovenkamp et al., J. Immunology 196 (2016) 1435-1441, conclude that with a significant effect on stability, half-life and binding characteristics of antibodies, IgG Fab glycosylation is an important—yet poorly understood—process. mAb glycosylation is complex and thus accurate, reproducible glycoprofiling can be challenging, particularly when this involves controlling glycoform fidelity at the Fc and Fab sites.

Glycosylation patterns can change with different expression systems, culture conditions, processes and scales of manufacture and optimisation of those is required in order to obtain and maintain desired glycosylation patterns. Understanding the interplay between cell growth, cell metabolism, IgG synthesis and glycosylation and how these factors vary among different cell lines and media compositions at the metabolic level will benefit bioprocess optimization.

Tachibana et al., Cytotechnology 16 (1994) 151-157 worked with an antibody produced by C5TN cells that is specific to lung adenocarcinoma and cross-reactive to Candida cytochrome C, whose unique feature is an N-glycosylation at the hypervariable region of the light chain. They discovered that in glucose limited conditions, the cell is less able to fully glycosylate the protein, with smaller than normal glycoforms being produced. The authors thus recommended continuous perfusion culture rather than conventional batch culture.

Hossler et al., Glycobiology (2009) 19(9) 936-949 describe an intricate relationship between variables arising from cellular media and process effects and control of protein glycosylation and Ehret et al., Biotechnol & Bioeng (2019) 116 816-830 describe the impact of cell culture media on protein glycosylation.

Fan et al., Biotech & Bioeng (2015) 112(3) 521-535 found that, in fed-batch processes for CHO cells with two chemically defined proprietary media, the balance of glucose and amino acid concentration is important for cell growth, IgG titer and Fc situated N-glycosylation such that faster cell growth, higher integral for viable cells and IgG production—and more mature glycoproteins—could be obtained as a result of a more balanced amino acid concentration in the culture and higher glucose consumption. The authors decided that the effect of media and process optimisation on Fc glycosylation should be understood from case-to-case and results from the interplay of the protein processing rate, cell metabolism and expression and activity of Golgi resident proteins.

Huang et al., Anal. Biochem. 349 (2006) 197-207 analyzed the effect of glycosylation on antibody clearance of an anti-AB-IgG1 monoclonal antibody and found rapid clearance of N-acetyl glucosamine terminated biantennary complex type glycans.

St Amand et al., Biotechnol Bioeng (2014) 111(10) 1957-70 found that each of manganese, galactose and ammonia when used as media supplements had significant effects on certain glycans and glycan distribution.

EP 1960428 is directed to antibodies against amyloid beta, with glycosylation in the variable region, to clear existing and prevent formation of new β-amyloid plaques in humans. The glycosylation in the V_{H} region is selected from a sugar structure of the bianntenary complex type without core fucosylation; of the bianntenary hybrid type; or of the bianntenary oligomannose type, wherein the hybrid and oligomannose structures are considered minor and, combined, are present at 25% or less of the composition. The oligomannose sugar structures are characterised by containing Man4, Man5 or Man6 subunits, including those mannose units in the typical N-linked core structure. There is no disclosure of either the need, or methods, for regulating the relative high mannose content in the antibody glycoforms.

The present invention has been devised in light of the above considerations.

### SUMMARY OF THE INVENTION

The present invention relates to monoclonal antibody compositions in which the relative content of a particular glycoform, the high mannose glycoform, at the N-glycosylation site in the Fab region of the antibody, is regulated. Diagnosis and methods of treatment of diseases with such antibody compositions and the medical uses of such antibody compositions are also provided, as well as methods for the preparation of such antibody compositions and the antibody compositions produced thereby.

The following are aspects of the invention in its broadest sense. Alternative and preferred embodiments, in which the nature of the features of the invention are more closely defined, follow in the detailed description.

Throughout the following disclosure, for the sake of brevity the term “antibody” will be used to encompass monoclonal antibodies, polyclonal antibodies, multispecific antibodies, including bispecific antibodies, and antibody fragments (as hereinafter defined) so long as they exhibit the desired antigen-binding activity.

In its first aspect, the invention provides a composition comprising a monoclonal antibody having N-glycosylation in the Fab region(s) thereof, wherein relative to the total amount of glycosylated Fab in the composition, about 20% or less of Fab regions in the composition have an N-linked high mannose glycan.

The composition of the invention may be a pharmaceutical composition. Alternatively, the composition of the invention may be a cell culture supernatant obtainable during and/or after recombinant production of the antibody.

In one aspect, the present invention provides a method for reducing the rate at which an antibody clears from the circulation of an animal to which the antibody has been administered, which method comprises regulating the relative content of high mannose Fab glycoforms of a glycosylated monoclonal antibody in a composition comprising the antibody.

In one aspect, the present invention envisages a method for regulating the relative content of high mannose Fab glycoforms of a glycosylated monoclonal antibody comprised in the composition of the invention, which method comprises, over all or a part of the production phase of fermentation, optimising the concentration of glucose in a culture medium used for producing the glycosylated monoclonal antibody by fermentation therein of a eukaryotic cell expressing the monoclonal antibody.

Optimising the concentration of a carbohydrate source for the eukaryotic cell in the culture medium during all or a part of the production phase comprises maintaining an average concentration of the carbohydrate source in the culture medium during all or a part of the production phase that correlates with the desired relative content of high mannose Fab glycoforms resulting from the fermentation.

The method may further comprise a step of recovering the monoclonal antibody from the culture medium.

In the invention the “relative” content means the content of high mannose Fab glycoforms in the composition in relation to the content of all other Fab glycoforms of the monoclonal antibody in the composition.

In one embodiment of the invention, in the composition, the high mannose Fab glycoforms make up about 20% or less of the total Fab glycoforms of the monoclonal antibody. Thus, the invention provides a monoclonal antibody composition, the composition comprising N-linked glycosylated Fab region(s), wherein about 20% or less of the N-linked glycosylated Fab region(s) are the N-linked high mannose glycoform.

In the methods of the invention, to achieve about 20% or less of Fab high mannose glycoforms of the monoclonal antibody in the composition, glucose is the carbohydrate source and the concentration of glucose is optimised such that the average glucose concentration (optionally with the average calculated over day −7 to day 0 of the production phase) in the recombinant production of the monoclonal antibody is from about 0.50 g/L to about 18.00 g/L, preferably from about 1.50 g/L to about 14.00 g/L and more preferably from about 2.00 g/L to about 12.50 g/L.

In a further aspect, the present invention provides a monoclonal antibody composition obtainable by a method set out above. The monoclonal antibody composition may be a cell culture supernatant, or may be a pharmaceutical composition.

In a further aspect, the present invention provides a method of treatment of a disease comprising administering to a patient suffering from the disease a composition comprising a monoclonal antibody having N-glycosylation in the Fab region(s) thereof, wherein relative to the total amount of N-glycosylated Fab regions in the composition, about 20% or less of Fab regions in the composition have an N-linked high mannose glycan.

In a further aspect, the present invention provides a composition comprising a monoclonal antibody having N-glycosylation in the Fab region(s) thereof, wherein relative to the total amount of N-glycosylated Fab regions in the composition, about 20% or less of Fab regions in the composition have an N-linked high mannose glycan, for use in the treatment of a disease in an individual suffering therefrom.

In addition, the present invention provides a composition comprising a monoclonal antibody having N-glycosylation in the Fab region(s) thereof, wherein relative to the total amount of N-glycosylated Fab regions in the composition, about 20% or less of Fab regions in the composition have an N-linked high mannose glycan for use in the diagnosis of a disease.

In these aspects, the disease may be, for example, dementia, Alzheimer's Disease, motor neuropathy, Parkinson's Disease, amylotrophic lateral sclerosis (ALS), scrapie, HIV-related dementia, Creutzfeld-Jakob disease (CJD), hereditary cerebral haemorrhage, Down's syndrome and neuronal disorders related to ageing; and cancer, such as metastatic colorectal cancer, metastatic non-small cell lung cancer, ovarian cancer and head and neck cancer.

### DESCRIPTION OF THE FIGURES

FIG. 1 Fab Glycosylation Species. The species are summed up into several sum parameters as Sum Fab hybrid mannose (Hybrid man), Sum Fab Sialylation (Sial), Sum Fab Galactosylation (Gal), Fab high mannose (High man) and Fab mannose (Man).

FIG. 2: Impact of glucose on Fab high mannose production, shown as area % Fab high mannose over average glucose concentration from day −7 to day 0 [g/L]. Available data from all representative runs of the given project shown (not only runs dedicated to glucose variations). Fermentation runs vary in scales, Roche production sites and minor process changes (e.g. agitation, aeration, cell bank, cell age). A glucose solution is added on demand via bolus or continuous addition.

FIG. 3A & 3B: Impact of glucose on Fab high mannose production. Fab high mannose as sum and also the 3 parts of the sum (Fab mannose 5, 6 and 7) in area % over average glucose concentration from day −7 to day 0 [g/L] (FIG. 3A). Comparison of different glucose average levels. One experimental setup dedicated for glucose variation, all other parameters are kept constant. Glucose is added on demand daily via bolus addition. Additionally, in FIG. 3B, the calculation of the glucose average is explained: the calculation is done based on the daily measured glucose concentration from samples before the bolus addition and the calculated glucose concentration after the bolus addition.

FIG. 4A & 4B: A comparison of the pharmacokinetics of Gantenerumab produced with a previous process (G3 process/highmannose high) with Gantenerumab produced according to the method of the invention (G4 process/highmannose low) in a clinical study. FIG. 4A is a linear scale and FIG. 4B is a semi-log scale.

FIG. 5: Plasma concentrations of total Gantenerumab (i.e. the sum of all Gantenerumab species in the material) and Gantenerumab with Man5/Man6 Fab glycans were determined following intravenous administration of Gantenerumab to rats (15 mg/kg) (for determination of Gantenerumab Man5/Man6 Fab glycans plasma concentrations see Example 4/Rat, intravenous Gantenerumab injection study 3).

FIG. 6A & 6B: Fab glycoform % in Gantenerumab produced according to the methods herein (highmannose low/G4 process). A comparison of the Man5, Man6, Man7 and Man8 content can be made with the Gantenerumab produced by a different method (highmannose high/G3 process)—FIG. 6B. Bulk data shown (fully purified by several purification steps).

FIG. 7: Pharmacokinetic data comparing results obtained with Gantenerumab produced according to the methods herein (G4 process) with Gantenerumab produced according to previous methods (G1, G2 or G3 processes). Data from bulk samples (fully purified by several purification steps). High mannose data from Fab glycoforms.

FIG. 8A & 8B: Clearance in rats is not affected when Man5 Fc glycoforms of glycosylated Gantenerumab are administered to rats (FIG. 80A, Panel B), whereas Man5 and Man6 levels in Fab glycosylation result in rapid clearance (FIG. 8B, Panels C and D). Data from PK rat study with G2 process-prepared material.

FIG. 9: Chromatogram illustrating the Fab glycan peaks from which the percentage of glycoforms can be calculated.

### DEFINITIONS

In order that the present invention may be more readily understood, certain terms are first defined.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless otherwise defined herein, scientific and technical terms used in connection with the present invention shall have the meanings commonly ascribed to them in the art. The meaning and scope of the terms should be clear, however, and in the event of any latent ambiguity, definitions provided herein take precedent over dictionary or extrinsic definition. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “anti-human A-beta antibody” and “an antibody specifically binding to human A-beta” refer to an antibody that is capable of binding the human A-beta (Aβ) peptide with sufficient affinity such that the antibody is useful as a diagnostic and/or therapeutic agent in targeting an A-beta peptide. It is of note that human A-beta peptide has several naturally occurring forms, whereby the human forms are referred to as Aβ39, Aβ40, Aβ41, Aβ42 and Aβ43. The most prominent form is Aβ42. The terms “anti-human A-beta antibody” and “an antibody specifically binding to human A-beta” also encompass antibodies that bind to a shortened fragment of the human A-beta polypeptide. A-beta peptide is also known as amyloid-beta or Aβ peptide and these peptides are a major constituent of amyloid plaques in the brains of individuals with Alzheimer's disease.

The term “antibody” herein is used in the broadest sense and encompasses various antibody structures, including but not limited to monoclonal antibodies, polyclonal antibodies, multi-specific antibodies (e.g., bispecific antibodies), and antibody fragments so long as they exhibit the desired antigen-binding activity. An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

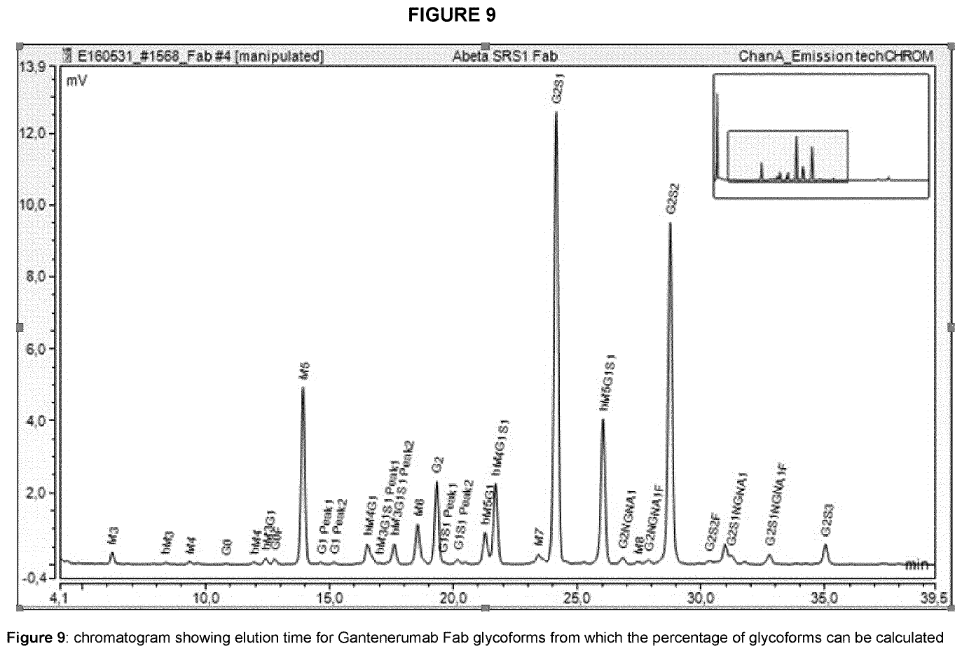

“Area %”, “Area % Fab” or “% Fab region” refers to the percentage of each glycoform (i.e. high mannose glycan or other glycoform) calculated e.g. from a chromatogram of separated glycans. FIG. 9 illustrates such a chromatogram. To calculate the percentage of M5-M7 Fab glycoforms, a baseline is drawn, e.g. in FIG. 9 from 5 to 39 minutes and then the area under the curve with that baseline is calculated (A1). The areas of the peaks M5-M7 are then divided by A1 and multiplied by 100 to give the percent M5-M7 (area % or %). In this way the total amount of glycosylated Fab having an N-linked high mannose glycan in a composition is calculated—e.g. about 20% or less of Fab regions in a composition having N-linked high mannose glycan.

“Average glucose concentration” refers to an average of the glucose concentration in the culture medium over the length or a part of the length of the culture process. The average may be calculated using the formulae described below. Glucose consumption by cells and additions to the medium, the glucose concentration in the initial medium, as appropriate, and the duration of the fermentation or the part thereof are all taken into account in determination of the average glucose concentration. The average glucose concentration can be determined by routine, e.g. daily, measurement of glucose concentration over the culture process and calculation therefrom, or can be assumed from previous fermentation runs under the same or similar conditions and set accordingly. The association between glucose concentration and the production of antibodies with a high mannose structure in the Fab region(s) described below can inform the selection of an average glucose concentration for a fermentation run. Glucose concentrations are given in g/L within this disclosure, which means pure glucose and not glucose monohydrate etc.

“Biomass” as used herein refers to the quantity or weight of cultured cells in the culture medium. Biomass may be measured directly or indirectly by determining viable cell density, total cell density, cell time integral (for viable and total cell density), cell volume time integral (for viable and total cell density), packed cell volume, dry weight or wet weight.

“Bioreactor” as used herein refers to any vessel used for the growth of a mammalian cell culture. Typically a bioreactor will be at least 0.25 litres and may be 1, 10, 100, 250, 500, 1000, 2500, 5000, 8000, 10,000, 12,000 litres or more, or any volume in between. The internal conditions of the bioreactor, including but not limited to pH, dissolved oxygen and temperature, are typically controllable during the culture period. A bioreactor can be composed of any material that is suitable for holding mammalian cell cultures suspended in media under the culture conditions of the present invention, including glass, plastic or metal or a combination thereof. A bioreactor may be multi- or single-use, re-usable, disposable or re-cyclable.

“Carbohydrate source” as used herein is the energy required for growth of eukaryotic cells in culture. Typically the carbohydrate source is a monosaccharide selected from glucose, galactose, fructose and mannose but may also be a polysaccharide such as maltose or starch when the appropriate culture medium is selected.

“Cell” and “cell line” are used herein interchangeably and all such designations include progeny.

“Cell density” as used herein refers to the number of cells present in a given volume of medium.

“Cell viability” as used herein refers to the ability of cells in culture to survive under a given set of culture conditions or experimental variations. The term as used herein also refers to that portion of cells which are alive at a particular time in relation to the total number of cells, living or dead, in culture at that time.

Drug “clearance” as used herein is the volume of plasma cleared of a drug over a specified time period. Thus, the unit of measurement for drug clearance is volume/time or, when normalised to body weight, volume/time/body weight.

“Continuous feeding” as used herein refers to providing nutrients to a cell culture medium continuously over the full or a part of the period of the culture. The amount and composition of feed added can be adjusted as necessary during culture.

“Culture” or “cell culture” as used herein refers to a cell population that is suspended in a medium under conditions suitable for survival and/or growth of the cell population. These terms will also be applied to the combination of the medium and cell population suspended therein.

“Culture conditions” and “fermentation conditions” are used herein interchangeably and are those conditions that must be satisfied to achieve successful cell culture and glycoprotein production. Typically these conditions include provision of an appropriate medium, as well as control of e.g. temperature, which should be about 37° C., but could also include a temperature shift during culture (e.g. 37° C. to 34° C.) and pH, which is normally between 6.8 and 7.2, as well as the provision of oxygen and carbon dioxide. Such conditions also include the manner in which the cells are cultured, e.g. shaker or robotic cultivation.

“Daily” when used in respect of a concentration, an amount or a measurement refers to a single 24 hour period. Thus a daily measurement means that e.g. the concentration of an element is measured once in a 24 hour period. A daily amount e.g. of an element added to the culture, is the total of additions of that element in a 24 hour period, and may include a single or multiple additions.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human anti-Abeta IgG heavy chain Fc region extends from Cys226, or from Pro230, to the carboxyl-terminus of the heavy chain. However, the C-terminal lysine (Lys447), or the C-terminal glycine (Gly446) and the C-terminal lysine (Lys447), of the Fc region may or may not be present. In one embodiment the anti-Abeta antibody as described herein is of IgG1 isotype and comprises a constant heavy chain domain of SEQ ID NO: 9. In one embodiment it comprises a constant heavy chain domain of SEQ ID NO: 9 without the C-terminal lysine (Lys447). In one embodiment it comprises a constant heavy chain domain of SEQ ID NO: 9 without the C-terminal lysine (Lys447) and without the C-terminal glycine (Gly446). Unless otherwise specified herein, numbering of amino acid residues in the Fc region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242.

“Fed-batch culture” as used herein refers to a method of culturing cells in which additional components are provided to the culture at a time or times subsequent to the beginning of the culture process. A fed-batch culture is typically stopped at some point and the cells and/or components in the medium are harvested and optionally purified.

“Galactosylated” as used herein in respect of glycoproteins, refers to glycoprotein comprising one or more galactose residues, resulting in G1 and G2 glycostructures.

“Glycan” refers to a polysaccharide or monosaccharide moiety: glucose (Glc), galactose (Gal) mannose (Man), fucose (Fuc), N-acetylgalactosamine (GalNAc), N-acetylglucosamine (GlcNAc) and sialic acid (e.g. N-acetylneuraminic acid (NANA or NeuNAc, where Neu is neuraminic acid). The processing of the sugar groups occurs co-translationally in the lumen of the ER and continues in the Golgi apparatus for N-linked glycoproteins. The Aβ N-glycans herein are N-glycosylated at the amide nitrogen of the asparagine (Asn) at position 52 in the variable region of the heavy chain of one or both antigen binding sites.

All N-linked oligo-/polysaccharides have a common “pentasaccharide core” of Man₃GlcNAc₂. The pentasaccharide core is also referred to as the “trimannose core”.

N-glycans differ from each other with respect to the presence of, and/or in the number of branches (also called antennae) comprising peripheral sugars such as GlcNAc, Gal, GalNAc, NANA and Fuc that are added to the pentasaccharide core structure. Optionally this structure may also contain a core fucose molecule and/or a core xylose molecule. For a review of standard glycobiology nomenclature see Essentials of Glycobiology Varki et al., CSHL Press (1999).

N-glycans are classified according to their branched constituents (e.g. oligomannose-type, complex or hybrid). Galactosylated species, with one or more terminal Gal residues on the core, include the G0, G1 and G2 species. Oligomannose N-glycans can be categorised herein as “mannose-type” or “high-mannose type”. A high-mannose type N-glycan has five or more mannose residues per glycan (including any forming the core), e.g. M5, M6 or M7. When there are only 3 or 4 mannose residues, e.g. M3 or M4, the glycan is a mannose-type. A complex-type N-glycan typically has at least one GlcNAc attached to the 1,3-mannose arm and at least one GlcNAc attached to the 1,6 mannose arm of a pentasaccharide core. Complex-type N-glycans may also have Gal or GalNAc residues that are optionally modified with NANA or other sialic acid derivatives. Complex-type N-glycans may also have intra-chain substitutions comprising “bisecting” GlcNAc, and core Fuc. Otherwise, or in addition, they may also have multiple antennae on the pentasaccharide core and are, therefore, also referred to as “multiple antennary-type glycans”. A hybrid-type N-glycan comprises at least one GlcNAc on the 1,3 mannose arm of the pentasaccharide core and one or more mannoses on the 1,6 mannose arm of the core. Terminal sialic acid residues may also be present. Hybrid mannose species thus include hM3, hM4, hM3G1, hM4G1, hM5G1, hM5G1S1, hM4G1S1 and hM3G1S1. Sialylated species include one or more terminal sialic acid residues, such species including G1S1, G2S1, G2S2 and the three hybrid mannose species hM5G1S1, hM4G1S1 and hM3G1S1.

Oligomannose structures (high-mannose type) include “M5”, “Man5” or “Man5 glycan”; “M6”, “Man6” or “Man6 glycan”; “M7”, “Man7” or “Man7 glycan”; “M8”, “Man8” or “Man8 glycan” and “M9”, “Man9” or “Man9 glycan”. “High mannose” refers to the amount or level of mannosylation, or mannosylated N-glycans and includes, for example, Man5, Man6, Man7, Man8 and Man9. In the present invention, “high mannose” is intended to include one or a mixture comprising the Man5, Man6 and Man7 glycoforms, although traces of Man8 or Man9 may also be present. All mannose residues in the glycan are counted, including any forming part of the core structure.

“Glycoprotein” refers to a protein or polypeptide having at least one glycan moiety.

The term “glycoform” refers to an isoform of a protein, e.g. an antibody that differs only with respect to the number and/or type of attached glycan(s). Glycoproteins often consist of a number of different glycoforms. A “high mannose glycoform” is an antibody in which the N-linked glycan at one or both Fab regions has a “high mannose” content, i.e. includes one or a mixture of Man5, Man6 and Man7 (optionally with traces of Man8) glycans. Embraced by the present invention are variants of such high mannose glycoforms identified e.g. using different glycoanalysis methods.

The terms G1, G2, G3 and G4 are used herein to describe process versions for the production of greater and lesser amounts of Gantenerumab in high mannose form. G2 and G3 processes are “high mannose high” process versions—i.e. higher amounts of the high mannose version are produced, for example up to 8% Fab high mannose for fully purified material, and G1 and G4 processes are “high mannose low” process versions, i.e. lower amounts of the high mannose version are produced, for example from 0-8% Fab high mannose for fully purified material. The method described herein is a G4 process.

“Host cell”, “host cell line” and “host cell culture” are used herein interchangeably and denote any kind of cellular system which can be engineered to generate glycoproteins. They refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

“Medium”, “cell culture medium” and “culture medium” are used herein interchangeably and refer to a solution containing nutrients which sustain growth of mammalian cells and production of glycoprotein therefrom. Typically such solutions provide essential and non-essential amino acids, vitamins, energy sources, lipids and trace elements required by the cell for minimal growth and/or survival. Such a solution may also contain supplementary components that enhance growth and/or survival above the minimal rate including, but not limited to, hormones and/or other growth factors, particular ions, such as sodium, chloride, calcium, magnesium and phosphate, buffers, vitamins, nucleosides or nucleotides, trace elements, amino acids, lipids and/or glucose or other energy source. A medium is advantageously formulated to a pH and salt concentration optimal for cell survival and proliferation. A medium may be a reduced serum or serum free medium, i.e. wherein the medium contains about 1-5% serum or when the medium is essentially free of any mammalian serum (e.g. fetal bovine serum), respectively. By “essentially free” of serum is meant that the medium comprises between 0-5% serum, preferably between about 0-1% serum and most preferably about 0-0.1% serum. A serum-free defined medium may be used, where the identity and concentration of each of the components of the medium is known. A medium may be a protein-free medium, i.e. this will contain no protein but will contain undefined peptides e.g. from plant hydrolysates. Media could include human serum albumin and human transferrin but potentially animal-derived insulin and lipids, or a medium containing human serum albumin, human transferrin, human insulin and chemically defined lipids. Alternatively, a medium may be a chemically-defined medium, i.e. a medium wherein all substances are defined and present in defined concentrations. These media could contain only recombinant proteins and/or hormones or a protein-free chemically defined medium, i.e. containing only low molecular weight constituents and synthetic peptides/hormones if required. Chemically defined media could also be completely free of any protein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “mono-glycosylated antibody” is an antibody comprising an N-glycosylation in one heavy (V_{H})-region of an individual antibody molecule. Such an antibody could be, e.g. Gantenerumab. For example, the mono-glycosylated form of Gantenerumab comprises a glycosylation on one variable region of the heavy chain, e.g. at position Asn 52, as discussed below. This mono-glycosylated antibody may also comprise glycosylation in the well conserved glycosylation site in the Fc part, e.g. Asn 306.

A “double-glycosylated antibody” comprises N-glycosylation on both variable regions of the heavy (V_{H})-region. For example, the double-glycosylated form of Gantenerumab comprises N-glycosylation on both variable regions of the heavy chain, e.g. at position Asn 52, as discussed below. This double-glycosylated antibody may also comprise glycosylation in the well conserved glycosylation site in the Fc part, e.g. Asn 306.

The following disclosure is of a percentage of Fab regions in a composition having an N-linked high mannose glycan. This refers to the proportion of Fab regions having that desired structure, relative to the total N-glycosylated Fab regions in the preparation. The Fab regions may form part of a monoclonal antibody comprised in a composition, which may be mono- or double-glycosylated, or the composition may be of Fab regions per se. Thus, if about 20% of Fab regions in a composition have an N-linked high mannose glycan, then the remaining 80% of the Fab regions in the preparation are glycoforms with an N-linked glycosylation in the Fab region(s) having a structure other than high mannose glycan, wherein the nature of that structure is not important to the present disclosure. The percentage of each glycoform (e.g. high mannose or other than high mannose) in the preparation may be calculated e.g. from a chromatogram of the glycans produced. FIG. 9 illustrates such a chromatogram. To calculate the percentage of M5-M7 glycoforms from FIG. 9, a baseline is drawn from 5 to 39 minutes (i.e. the maximum elution time) and then the area under the curve with that baseline is calculated (A1). The area of the peaks M5-M7 is then divided by A1 and multiplied by 100 to give the percent M5-M7.

A “perfusion culture” when used herein refers to a cell culture process involving the constant feeding of fresh media and removal of spent media and product while retaining high numbers of viable cells. Cells are not removed during perfusion culture. Removal of spent media while keeping cells in culture can be done using alternating tangential-flow filtration (ATF) and standard tangential-flow filtration (TFF), or cells can be retained by binding them to a substrate in the bioreactor.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation or composition, other than an active ingredient, which is non-toxic to a subject. A pharmaceutically acceptable carrier includes, but is not limited to, a buffer, excipient, stabiliser or preservative.

“Protein” as used herein refers to one or more polypeptides that function as a discrete unit. When the protein contains only one polypeptide to function, the terms polypeptide and protein are interchangeable.

“Splitting” as used herein is also known as passaging or subculture of cells. This involves transferring a small number of cells into a fresh medium, whereby the split cells seed the new culture. In suspension cultures, a small amount of the culture containing a few cells is diluted into a larger volume of fresh medium.

“Therapeutic” as used herein relates to the treatment of disease with the intention of healing the disease. Therapeutic antibodies can activate, repress or alter endogenous immune responses to specific cells or molecules. Therapeutic monoclonal antibodies are used for the treatment of diseases such as autoimmune, cardiovascular and infectious diseases, cancer and inflammation.

The following abbreviations are used herein:
Aβ A-beta peptide
ADCC Antibody dependent cell cytotoxicity
AUC Area under the curve
CDC Complement dependent cytotoxicity
CDR Complementarity Determining Region
Complex-F GlcNAc GlcNAc Man₃ GlcNAc₂ Gal₀₋₂
Core GlcNAc₂ Man₃
Cmax maximum serum concentration after drug administration
CTI Cell time integral
ECLIA electrochemiluminescence immunoassay
ELISA Enzyme linked immunosorbent assay
Fab Antigen binding fragment
Fc Fragment Crystallizable Region
FR Framework Region
Fuc L-Fucose
G0 GlcNAc Fuc GlcNAc Man₃ GlcNAc₂
G0-F GlcNAc GlcNAc Man₃ GlcNAc₂
G1 GlcNAc Fuc GlcNAc Man₃ GlcNAc₂ Gal
G1-F GlcNAc GlcNAc Man₃GlcNAc₂Gal
G2 GlcNAc Fuc GlcNAc Man₃ GlcNAc₂ Gal₂
G2 1SA GlcNAc Fuc GlcNAc Man₃ GlcNAc₂ Gal₂ NANA₁
Gal D-Galactose
GlcNAc N-Acetylglucosamine
HILIC Hydrophilic interaction chromatography
HPLC High Performance Liquid Chromatography
High Mannose GlcNAc₂ Man₅₋₇
HVR Hypervariable region
ICP-MS Inductively coupled plasma mass spectrometry
i.v. intravenous
LDH Lactate dehydrogenase
mAb monoclonal antibody
Man D-Mannose
Man5/M5 GlcNAc₂ Man₅
Man6/M6 GlcNAc₂ Man₆
Man7/M7 GlcNAc₂ Man₇
Man8/M8 GlcNAc₂ Man₈
MCB Master Cell Bank
NANA N-Acetylneuraminic acid
PSB Primary Seed Bank
RSME Root mean-square-error
s.c. subcutaneous
UHPLC Ultra-High Performance Liquid Chromatography
Vss Average apparent Volume of distribution
WCB Working Cell Bank

### DETAILED DESCRIPTION

Embodiments and experiments illustrating the principles of the invention will now be discussed with reference to the accompanying figures.

1. Fab Compositions

According to this aspect, the present invention provides a composition comprising a monoclonal antibody having N-glycosylation in the Fab region(s) thereof, wherein relative to the total amount of glycosylated Fab in the composition, about 20% or less of Fab regions in the composition have an N-linked high mannose glycan in the Fab region(s) thereof. The composition may be either a cell culture supernatant or a pharmaceutical composition.

Compositions of the invention may comprise, contain or consist of the monoclonal antibody. In the composition the monoclonal antibody is substantially pure, in that antibodies with differing specificity are typically not present. Components other than the monoclonal antibody may also be present in the composition, as discussed in more detail below.

The following passages are also relevant to the antibody composition produced by the method of a further aspect of this invention.

In a preferred embodiment of this aspect of the invention, in the composition comprising a monoclonal antibody, the percentage of Fab regions having N-linked high mannose glycan is from about 0-20%, more preferably from about 0-15%, or about 0-12% and yet more preferably from about 0-10%. As stated above, this percentage of Fab regions having N-linked high mannose glycan is relative to the total N-linked glycosylated Fab regions. In a further preferred embodiment, in the composition, the percentage of Fab regions having N-linked high mannose glycan is about 15% or less, preferably about 12% or less, and yet more preferably about 10% or less. In a further preferred embodiment, in the composition the percentage of Fab regions having N-linked high mannose glycan is more than about 2%, yet more preferably more than about 4%. In a further preferred embodiment, in the composition comprising a monoclonal antibody, the percentage of Fab regions having N-linked high mannose glycan is from about 2-20%, or about 2-15%, or about 2-12% or about 2-10% or from about 4-20%, about 4-15%, about 4-12% or about 4-10%.

In this aspect of the invention, about 20% or less of Fab regions in the composition comprising a monoclonal antibody have an N-linked high mannose glycan. A high mannose glycan may be a glycan having from 5 to 9 mannose residues in total. The “total” as used herein includes the mannose residues forming part of the core structure (GlcNAc₂ Man₃). The high mannose glycan may be a one of or a mixture of any two or more glycans having from 5 to 9 mannose residues in total. In other words, the composition comprising a monoclonal antibody may comprise a number of Fab regions with a high mannose glycan having 5 mannose residues, a number of Fab regions with a high mannose glycan having 6 mannose resides, a number of Fab regions with a high mannose glycan having 7 mannose residues, a number of Fab regions with a high mannose glycan having 8 mannose residues and/or a number of Fab regions with a high mannose glycan having 9 mannose residues. In some cases, there may be no 8 or 9 mannose glycoforms. The number of each high mannose glycoform (i.e. M5, M6, M7, M8 and M9) in the composition comprising a monoclonal antibody is not important to the present invention, so long as the relative content of high mannose Fab regions in the composition, i.e. relative to the total number of glycosylated Fab regions in the composition, is about 20% or less.

Typically the high mannose glycan thus has from 2 to 6 mannose residues attached to the GlcNAc₂ Man₃ core. The high mannose glycan may thus be Man5 (GlcNAc₂ Man₅); Man6 (GlcNAc₂ Man₆); Man7 (GlcNAc₂ Man₇); Man8 (GlcNAc₂ Man₈) or Man9 (GlcNAc₂ Man₉). A high mannose glycan of the present disclosure may be one or a mixture of Man5, Man6, Man7, Man8 and Man9 glycoforms.

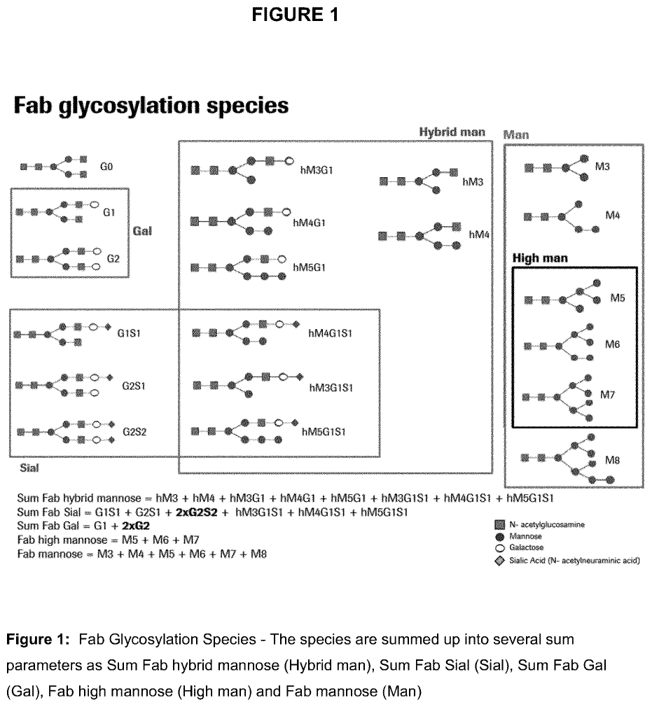

In a preferred embodiment of the present invention, the high mannose glycan is one or a mixture of Man5, Man6 and Man7 glycoforms. Typically, each of Man5, Man6 and Man7 glycoforms is present in the high mannose glycan composition. These glycoforms are illustrated in FIG. 1. Negligible amounts (i.e. 0.1% or less) of a Man8 or Man9 glycoform may be present. Typically neither Man8 nor Man9 are present in any significant amounts in this embodiment.

The relative amount of each high mannose glycoform, e.g. each of Man5, Man6 and Man7, in the glycosylated Fab regions of the monoclonal antibody in the composition is not important, so long as the total of the high mannose glycoforms in the glycosylated Fab regions of the monoclonal antibody in the composition is about 20% or less or falls within the percentage ranges set out above. Thus, the present invention contemplates in the composition comprising a monoclonal antibody from about 0-10%, about 0-12%, about 0-15% or about 0-20% of Fab high mannose glycan comprising one or more of Man5, Man6 or Man7, preferably either from about 2-10%, about 2-12%, about 2-15% or about 2-20% or about 4-10%, about 4-12%, about 4-15% or about 4-20%, of a Fab high mannose glycan comprising one or more of Man5, Man6 or Man7. By “one or more” in this respect is meant one, two or three of Man5, Man6 and Man7, wherein the combinations can be Man5 and Man6, Man5 and Man7, Man6 and Man7 or Man5, Man6 and Man7.

In this aspect of the invention, less than 80%, preferably less than about 85%, about 88% or about 90% and more preferably from about 80% to about 98%, about 85% to about 98%, about 88% to about 98% or about 90% to about 98%, or about 80% to about 96%, about 85% to about 96%, about 88% to about 96% or about 90% to about 96% of the N-linked glycosylation in the Fab regions comprises a glycan structure that is other than high mannose and is selected from galactosylated, sialylated, hybrid mannose or mannose-type structures. The composition may contain a small percentage, e.g. less than about 5%, of antibodies not containing any N-linked glycosylation.

Hybrid mannose or mannose/mannose-type structures do not include the high mannose glycoforms described above. “Unidentified” glycan forms may also be present. The exact nature of the glycoforms that are not high mannose is not essential to the present invention. Typically such glycoform structures include G1, G2, G1S1, G2S1, G2S2, hM3G1, hM4G1, hM5G1, hM3, hM4, hM4G1S1, hM3G1S1, hM5G1S1, M3 and M4. These structures are illustrated in FIG. 1. In a preferred embodiment, less than 80% of the Fab regions in the composition comprising a monoclonal antibody have an N-linked glycan structure that is selected from galactosylated and sialylated. Particularly preferred such structures include G1, G2, G1S1, G2S1, G2S2, hM3G1S1, hM4G1S1 and hM5G1S1. The invention contemplates a monoclonal antibody composition comprising a monoclonal antibody having N-glycosylation in the Fab region(s) thereof, wherein relative to the total amount of glycosylated Fab regions in the composition, about 20% or less of Fab regions in the composition have an N-linked high mannose glycan and about 80% or more of the Fab regions in the composition have an N-linked galactosylated, sialylated, hybrid mannose or mannose glycan. The structures of the high mannose, galactosylated, sialylated, hybrid mannose and mannose/mannose-type glycans can be any one or more of the structures described herein.

In this aspect of the invention, the monoclonal antibody is a homogeneous population of antibodies specifically targeting a single epitope on an antigen. The monoclonal antibody is N-glycosylated in the Fab region(s) thereof. As is well known, a monoclonal antibody consists of one Fc fragment and two Fab fragments. The monoclonal antibody in the composition of this aspect of the invention may have N-glycosylation at either one or both of the antigen binding fragments, that is, the antibody may be mono-glycosylated or double-glycosylated. Thus in one embodiment, the monoclonal antibody in the composition of the invention is double glycosylated, that is it is N-glycosylated at both of the antigen binding fragments (Fab regions) and in another embodiment the monoclonal antibody in the composition is mono-glycosylated, that is it is N-glycosylated at only one of the antigen binding fragments (Fab regions). Compositions of the present invention may contain substantially pure mono-glycosylated, substantially pure double-glycosylated or a mixture of mono- and double-glycosylated antibodies.

The site within the Fab region at which N-glycosylation is present will depend on the monoclonal antibody in the composition. N-glycosylation typically occurs at an asparagine (Asn) in the variable region of the heavy chain (V_{H}) region. Potential glycosylation sites comprise the Asn-X-Ser/Thr motif in the amino acid sequence in the heavy chain(s) of the antibody and the monoclonal antibody comprised in the composition of the invention may either naturally contain such glycosylation sites or may be engineered to introduce such glycosylation sites to contribute to antibody diversification.

Monoclonal antibodies comprised in the composition of this aspect of the invention may be therapeutic or diagnostic antibodies, preferably therapeutic monoclonal antibodies. In a further embodiment, the antibody is a chimeric, humanized or fully human antibody.

In certain embodiments, an antibody provided herein is a chimeric antibody. Certain chimeric antibodies are described, e.g. in U.S. Pat. No. 4,816,567; and Morrison, et al., P.N.A.S. 81 (1984) 6851-6855. In one example, a chimeric antibody comprises a non-human variable region (e.g. a variable region derived from a mouse, rat, hamster, rabbit or non-human primate, such as a monkey) and a human constant region. In a further example, a chimeric antibody is a “class-switched” antibody in which the class or subclass has been changed from that of the parent antibody. Chimeric antibodies include antigen-binding fragments thereof.

In certain embodiments, a chimeric antibody is a humanized antibody. Typically a non-human antibody is humanized to reduce immunogenicity to humans, while retaining the specificity and affinity of the parental non-human antibody. Generally a humanized antibody comprises one or more variable domains in which HVRs, e.g. CDRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g. the antibody from which the HVR residues are derived) e.g. to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain.

Although compositions of this aspect of the invention typically contain whole antibodies, the invention also extends to antibody fragments which include, but are not limited to Fab, Fab′, Fab′-SH, F(ab′)₂, Fv, single-chain Fab (scFab); single-chain variable fragments (scFv) and single domain antibodies (dAbs), as well as to biosimilars.

In certain embodiments, an antibody provided herein is an antibody fragment comprising the Fab N-glycosylation site. The term “antibody fragment” refers to a molecule other than an intact antibody that comprises a portion of an intact antibody that retains the ability to specifically bind to an antigen. Antibody fragments include, but are not limited to Fab, Fab′, Fab′-SH, F(ab′)₂, Fv, single-chain Fab (scFab); single-chain variable fragments (scFv) and single domain antibodies (dAbs). For a review of certain antibody fragments, see Holliger and Hudson, Nature Biotechnology 23:1126-1136 (2005).

In one embodiment, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. Fab′ fragments differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another embodiment, the antibody fragment is a diabody, a triabody or a tetrabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further embodiment, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another embodiment, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another embodiment, the antibody fragment is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

In this disclosure an antibody fragment is other than an intact antibody, comprises the Fab N-glycosylation site and comprises a portion of an intact antibody that retains the ability to specifically bind to an antigen.

Antibody fragments can be made by various techniques, including but not limited to proteolytic digestion of an intact antibody as well as recombinant production by recombinant host cells (e.g., CHO), as described herein.

In certain embodiments, an antibody provided herein is a multispecific antibody, e.g., a bispecific antibody. Multispecific antibodies are monoclonal antibodies that have binding specificities for at least two different sites, i.e., different epitopes on different antigens or different epitopes on the same antigen. In certain embodiments, one of the binding specificities is for human A-beta and the other specificity is for any other antigen. In certain embodiments the other specificity is for the transferrin receptor, as described in EP 3356400. Such antibodies are useful for the diagnosis or treatment of Alzheimer's Disease. In one embodiment the binding specificity for human A-beta is provided by Gantenerumab or a part thereof. In certain embodiments, bispecific antibodies may bind to two (or more) different epitopes on A-beta. Multispecific (e.g., bispecific) antibodies may also be used to localize cytotoxic agents or cells to cells which express A-beta. Multispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

The bispecific antibody or antigen binding fragment thereof also includes a “Dual Acting Fab” or “DAF” comprising an antigen binding site that binds to a conformational epitope on A-beta as well as another different antigen, or two different epitopes of A-beta (see, e.g., US 2008/0069820 and WO 2015/095539).

The present invention relates to any monoclonal antibody with glycosylation in the Fab region(s) thereof, particularly therapeutic antibodies.

Therapeutic antibodies include Cetuximab, which has an N-glycosylation site in the V_{H} CDR2, containing an N-glycan at Asn 99 of the V_{H} region; Solanezumab, which has an N-glycosylation site in the V_{H} CDR2 and human A-beta antibodies such as Gantenerumab, which contains an N-glycan at Asn 52 of the V_{H} region.

Solanezumab is a humanized monoclonal IgG1 antibody directed against the mid-domain of the Aβ peptide. It recognizes soluble monomeric, not fibrillar, Aβ.

Cetuximab is an epidermal growth factor receptor (EGFR) inhibitor used in the treatment of metastatic colorectal cancer, metastatic non-small cell lung cancer and head and neck cancer. It is a chimeric monoclonal antibody distributed under the trade name Erbitux™.

Gantenerumab is a fully human IgG1 monoclonal antibody designed to bind with sub-nanomolar affinity to a conformational epitope on Aβ fibrils in the treatment of Alzheimer's Disease. Gantenerumab is also known as R04909832 and RG1450. Gantenerumab is described in EP 1960428 B1. In general, the heavy chain constant domain 2 (CH2) of the Gantenerumab IgG-Fc region is N-glycosylated through covalent attachment of oligosaccharide at asparagine 306 (corresponding to Asn 297 in the Kabat system). In addition Gantenerumab is N-glycosylated at asparagine 52 in CDR2 (SEQ ID NO:2) of the V_{H} (Fab) region.

In a preferred embodiment, the monoclonal antibody in the composition of the invention is a human A-beta antibody, preferably Gantenerumab.

The heavy chain of Gantenerumab comprises a V_{H} domain which comprises:
a CDR1 comprising the amino acid sequence of SEQ ID NO:1;
a CDR2 comprising the amino acid sequence of SEQ ID NO:2; and
a CDR3 comprising the amino acid sequence of SEQ ID NO:3.

The light chain of Gantenerumab comprises a V_{L} domain which comprises:
a CDR1 comprising the amino acid sequence of SEQ ID NO:4;
a CDR2 comprising the amino acid sequence of SEQ ID NO:5; and
a CDR3 comprising the amino acid sequence of SEQ ID NO:6.

In a particular embodiment, the V_{H} domain of Gantenerumab comprises the amino acid sequence of SEQ ID NO:7 and the V_{L} domain of Gantenerumab comprises the amino acid sequence of SEQ ID NO:8.

In a particular embodiment of the present invention, the heavy chain of Gantenerumab comprises the amino acid sequence of SEQ ID NO:9.

In a particular embodiment of the present invention, the light chain of Gantenerumab comprises the amino acid sequence of SEQ ID NO:10.

In a particularly preferred embodiment, the monoclonal antibody is Gantenerumab, a bi-specific antibody comprising Gantenerumab, or a fragment of Gantenerumab comprising a glycosylated Fab region and which retains the ability to bind the antigen. In these embodiments, the monoclonal antibody has a V_{H} and a V_{L} CDR amino acid sequences as set out in SEQ ID Nos:1-6, above, a V_{H} and a V_{L} domain amino acid sequences of SEQ ID NO:7 and SEQ ID NO:8, or a heavy and light chain comprising the amino acid sequence of SEQ ID NO:9 and 10.

Thus, the present invention provides a composition comprising a monoclonal antibody comprising a V_{H} CDR1 comprising the amino acid sequence of SEQ ID NO:1; a V_{H} CDR2 comprising the amino acid sequence of SEQ ID NO:2; a V_{H} CDR3 comprising the amino acid sequence of SEQ ID NO:3; a V_{L} CDR1 comprising the amino acid sequence of SEQ ID NO:4; a V_{L} CDR2 comprising the amino acid sequence of SEQ ID NO:5; and a V_{L} CDR3 comprising the amino acid sequence of SEQ ID NO:6, said composition comprising about 20% or less of a high mannose Fab glycoform relative to the total amount of V_{H} glycosylated Fab regions in the composition, wherein said glycosylation is N-glycosylation at Asn52 in the CDR2 of the antibody.

Alternatively, or in addition, the present invention provides a composition comprising a monoclonal antibody comprising a V_{H} domain comprising the amino acid sequence of SEQ ID NO:7; and a V_{L} domain comprising the amino acid sequence of SEQ ID NO:8; said composition comprising about 20% or less of a high mannose Fab glycoform of said antibody relative to the total amount of V_{H} glycosylated Fab regions in the composition, wherein said glycosylation is N-glycosylation at Asn52 in SEQ ID NO:7.

Alternatively, or in addition, the present invention provides a composition comprising a monoclonal antibody comprising a heavy chain comprising the amino acid sequence of SEQ ID NO:9; and a light chain comprising the amino acid sequence of SEQ ID NO:10; said composition comprising about 20% or less of a high mannose Fab glycoform of said antibody relative to the total amount of V_{H} glycosylated Fab regions in the composition, wherein said glycosylation is N-glycosylation at Asn52 in SEQ ID NO:9.

In any, or all, of the above aspects, the monoclonal antibody Gantenerumab may also be N-glycosylated in the Fc region thereof.

In an alternative preferred embodiment, the monoclonal antibody is a bi-specific antibody comprising Gantenerumab. In this embodiment, the bispecific antibody comprises an additional Fab fragment which binds to a human transferrin receptor. In this embodiment, the bispecific antibody comprises:
a heavy chain with the amino acid sequence of SEQ ID NO:9;
a light chain with the amino acid sequence of SEQ ID NO:10;
a heavy chain Fab fragment with an amino acid sequence of SEQ ID NO:11; and
a light chain with an amino acid sequence of SEQ ID NO:12.

Various studies have shown that the nature of the glycosylation profile, such as the relative content of a high mannose glycoform N-linked to the Fab region of a therapeutic or diagnostic monoclonal antibody, has an impact on the pharmacokinetics (PK). In particular, animal studies have demonstrated that there is rapid clearance in vivo of Fab high mannose species. This is shown in FIG. 10.

The “clearance” of a mAb in vivo will determine the body's “exposure” to the mAb—which will in turn determine the extent of the pharmacodynamic (PD) effects of the antibody. The exposure-response (PK-PD) relationship determines the outcome of a drug's effects on the body. Previous studies have suggested that glycosylation at the Fc region of an antibody may be relevant for PK, with binding of the glycan to its receptor causing glycan-mediated clearance and tissue distribution. Glycan receptors that have been attributed to the removal of glycoproteins in vivo include the mannose receptor (ManR) and the asialoglycoprotein receptor (ASGPR), both of which are carbohydrate-specific endocytic receptors. As reported by Liu et al., J. Pharmaceutical Sciences (2015) 104:1866-1884, glycosylated mAbs (with Fc glycosylation only) with mAbs deliberately manufactured to have terminal high mannose glycans, have been shown to be rapidly cleared from the blood and localised in the liver.

By controlling the relative amount of mannose in the V_{H} N-linked glycans of monoclonal antibodies, and in particular of human A-beta antibodies such as Gantenerumab, the present inventors have demonstrated that they can control antibody clearance and thus exposure of the body to the effects of the antibody. The production of a homogeneous population of antibodies in which the relative amount of a specific glycoform at the Fab N-glycosylation site(s) is controlled thus results in a therapeutic with a consistent pharmacological profile. In other words, regulating the Fab glycoform distribution affects the pharmacological profile of an antibody.

In one embodiment herein, Gantenerumab produced according to the methods herein, in particular Ganternerumab having from about 2-10%, preferably from about 4-10% high mannose, stays longer in the circulation and has a better bioavailability than Gantenerumab with a higher relative amount of high mannose glycans, typically produced according to a different method. As is illustrated in the examples herein, an increase in bioavailability in humans following sc administration of Gantenerumab of about 18% can be achieved using the antibody in which the relative amount of high mannose (M5-M7) at the Fab N-glycosylation site is between about 5 and 6%, e.g. when the antibody is produced according to the methods herein, as compared to Gantenerumab produced according to a different method in which the relative content of high mannose is about 13% (Man5-7).

In one aspect, the present invention provides a method for reducing the rate at which an antibody clears from the circulation of an animal to which the antibody has been administered, which method comprises regulating the relative content of high mannose Fab glycoforms of a glycosylated monoclonal antibody in a composition comprising the antibody.

Any or all of the following features may be taken alone or in combination in this aspect of the invention:
the animal is a mammal, preferably a human; and/or
the monoclonal antibody is an anti human A-beta antibody; and/or
the monoclonal antibody is Gantenerumab or bi-specific Gantenerumab, preferably wherein the bispecific Gantenerumab has binding specificity for A-beta and the transferrin receptor; and/or
the relative content of high mannose glycoform in the antibody composition is about 2-10%, preferably 4-10% and more preferably 5-9%; and/or
the antibody is produced according to a method described herein; and/or
bioavailability of the antibody increases by about 18% as compared to the same antibody with a relative content of the high mannose glycoform of about 13%, typically produced according to a different method.

Techniques for the determination of glycan primary structure are well known and are described in detail, for example, in Montreuil, Polysaccharides in Medicinal Applications (1996) 273-327. It is therefore a routine matter for one or ordinary skill in the art to isolate a population of peptides produced by a cell and determine the structure(s) of the glycans attached thereto. For example, efficient methods are available for (i) the splitting of glycosidic bonds either by chemical cleavage such as hydrolysis, acetolysis, hydrazinolysis, or by nitrous deamination; (ii) complete methylation followed by hydrolysis or methanolysis and by gas-liquid chromatography and mass spectroscopy of the partially methylated monosaccharides; and (iii) the definition of anomeric linkages between monosaccharides using exoglycosidases, which also provide insight into the primary glycan structure by sequential degradation. Fluorescent labelling and subsequent high performance liquid chromatography (HPLC), e.g. normal phase HPLC (NP-HPLC), mass spectroscopy and nuclear magnetic resonance (NMR) spectrometry, e.g. high field NMR, may also be used to determine the primary glycan structure.

Kits and equipment for carbohydrate analysis are also commercially available. Fluorophore Assisted Carbohydrate Electrophoresis (FACE) is available from Glyko. Inc. (Novato, California). In FACE analysis, glycoconjugates are released from the peptide with either Endo H or N-glycannase (PNGase F) for N-linked glycans. The glycan is then labelled at the reducing end with a fluorophore in a non-structure discriminating manner. The fluorophore labelled glycans are then separated in polyacrylamide gels based on the charge/mass ratio of the saccharide as well as the hydrodynamic volume. Images are taken of the gel under UV light and the composition of the glycan is determined by the migration distance as compared with standards. Oligosaccharides can be sequenced in this manner by analysing migration shifts due to the sequential removal of saccharides by exoglycosidase digestion.

Methods described herein embrace the production of glycosylation variants, e.g. identified by methods other than those described herein, so long as those variants fall under the definitions herein of high mannose.

For antibodies with both Fc and Fab glycosylation, analysis of the relative distribution of the N-glycans can comprise cleaving Fc glycans from the antibody backbone using e.g. endoglycosidase PNGaseF and separating the released carbohydrate from the protein by ultrafiltration. Fab glycans can then be released by rapid PNGaseF digestion and also separated from the protein by ultrafiltration. Fc and Fab glycans can be labelled separately, e.g. using 2-aminobenzamide, and then analysed by HILIC UHPLC (hydrophilic interaction chromatography/ultra-high performance liquid chromatography) with fluorescence detection.

The monoclonal antibodies comprised in the composition of this aspect of the invention will typically also be N-glycosylated in the Fc region thereof. As a generality, Fc glycosylation occurs at Asn 297 of the C_{H}2 domain (or an equivalent conserved Fc-position Asn corresponding to Asn 297 in the Kabat system). The glycans in the Fc region are typically of a complex biantennary type and may comprise a heptasaccharide core with variable addition of outer arm sugars. In one example, the glycosylation in the Fc V_{H} region is selected from:
(a) a biantennary complex type structure without core fucosylation;
(b) a biantennary hybrid type; or
(c) a biantennary oligomannose type.

Control of Fc glycosylation is described in the prior art and is within the skill of the person in the art.

2. Producing High-Mannose Glycoforms of Mabs with N-Linked Glycosylation in the Fab Region

In the biopharmaceutical industry, fed-batch CHO cell culture is the most commonly used process for IgG production. As described by Fan et al., Biotechnol. Bioeng. (2015) 112(3) 521-535, amino acid and glucose consumption, cell growth, metabolism, antibody titre and N-glycosylation patterns in the IgG Fc region are always the major concerns during upstream process optimisation, with the balance of glucose and amino acid concentration in the culture being important for cell growth, IgG titer and N-glycosylation.

Without being bound by any particular theory, the present inventors have discovered that there is a relationship between the bioavailability of an expressed antibody and the content of the high mannose Fab glycoform of the antibody relative to total N-linked glycosylated Fab in an antibody composition. Furthermore, the present inventors have discovered that a desired relative high mannose content in the antibody glycan can be achieved by regulating the average concentration of the carbohydrate source for the cells in the culture medium during cultivation and growth of the cells and production of the antibody in culture.

As nutrients such as glucose are required for both growth of the recombinant cells and for glycosylation of the antibody produced, the balance of glucose in the culture medium over part or all of the fermentation process is very important. For example, if too much glucose is present, the cells may grow well, but the relative content of high mannose Fab glycoforms of the antibody produced may be so high as to have a detrimental effect on the bioavailability of the antibody composition. In another example, allowing significant or substantial fluctuations in the concentration of glucose over the culture period may also affect either or both of cell growth and relative glycan content in the expressed antibody. The present inventors have taken these factors into consideration when formulating the methods below.

As in the disclosure relating to Fab Compositions, the monoclonal antibody of the following aspects has N-linked high mannose glycans in the Fab region(s) thereof. Reference throughout the following disclosure to “high mannose Fab glycoforms” is, as with the earlier described Fab Compositions, to such glycoforms being N-linked.

In this aspect, the present invention provides a method for reducing the rate at which an antibody clears from the circulation of an animal to which the antibody has been administered, which method comprises regulating the relative content of high mannose Fab glycoforms of a glycosylated monoclonal antibody in a composition comprising the antibody.

In this aspect, the present invention also provides a method for increasing the bioavailability of a glycosylated monoclonal antibody in the circulation of an animal to which the antibody has been administered, which method comprises regulating the relative content of high mannose Fab glycoforms of a glycosylated monoclonal antibody in a composition comprising the antibody.

Typically, in the above method, the regulation will result in 20% or less of high mannose Fab glycoforms of a glycosylated monoclonal antibody in a composition comprising the antibody, relative to the total number of glycosylated Fab regions in the composition.

Typically, the rate at which an antibody in which the relative content of high mannose Fab glycoforms of the antibody is regulated according to the methods described herein clears from the circulation of an animal to which the antibody has been administered is reduced by at least about 4 percentage points, preferably at least about 5 percentage points, as compared to the same antibody produced by a method in which the relative content of high mannose Fab glycoforms in the antibody comprised in the composition is not regulated in the manner disclosed herein.

A method for regulating the relative content of high mannose Fab glycoforms of a glycosylated monoclonal antibody comprised in the composition of the invention may comprise, over the all or a part of the production phase of fermentation, optimising the concentration of the carbohydrate source for the eukaryotic cells in a culture medium used for producing the glycosylated monoclonal antibody by fermentation therein of a eukaryotic cell expressing the monoclonal antibody. The concentration of other nutrients in the culture medium may also be optimised.

This method may further comprise a step of recovering the monoclonal antibody from the culture medium.

In these embodiments of the invention, and as defined above, the “relative” content means the content of high mannose Fab glycoforms in relation to the content of all other Fab glycoforms of the monoclonal antibody in the composition. The content of glycoforms is typically expressed as a percentage, as set out above.

In one embodiment of the invention, in the composition the high mannose Fab glycoforms make up about 20% or less of the total Fab glycoforms of the monoclonal antibody. This is a preferred glycoform distribution.

Features of the antibody composition itself, i.e. the product of the above method, are as described in the previous section. Any or a mixture of the features of the monoclonal antibody composition itself are equally applicable to the product of the method of this aspect of the invention and the reader is referred to the above sections of the description to save on repetition thereof here.

In the following description, glucose is the exemplified carbohydrate source. However, any or a combination of glucose, galactose, fructose, mannose or maltose may be the source of carbohydrate for the cells in culture.

In a preferred aspect of the methods of the invention, the concentration of glucose in the culture medium is optimised in order to achieve the desired relative content of Fab high mannose glycoform of the monoclonal antibody in the composition (desired glycoform distribution). In one aspect, optimisation of the glucose concentration involves monitoring and controlling the glucose concentration in the culture medium. Monitoring and controlling of the concentration of glucose and optionally one or more other nutrients in the culture medium may be performed throughout the culture process, or during a part of the culture process or may take place during one or more phases of the culture process, typically only during the production phase or a part thereof, as necessary. In an alternative case, the average glucose concentration and optionally concentrations of other nutrients may be optimised based on experience, e.g. from earlier fermentations, to achieve the desired glycoform distribution. In that case, monitoring of the concentrations through all or a part of the fermentation/production process may not be required.

In a preferred aspect of the methods of the invention, the concentration of glucose in the culture medium is optimised in order to achieve the desired, optionally the preferred, glycoform distribution.

In the above aspects, the concentrations are optimised over all or a part of the fermentation process, typically all or a part of the growth and/or production phases. In one case the glucose concentration is optimised over all of the growth phase or over a part of the growth phase. In one case the glucose concentration is optimised over all of the production phase or a part of the production phase. In one case the glucose concentration is optimised over all or a part of the growth phase and all or a part of the production phase.

In the methods of the invention and/or the preferred aspects set out above, to achieve about 20% or less of Fab N-linked high mannose glycoforms of the monoclonal antibody in the composition, the average amount of glucose in the culture medium in the production phase of the culture process in the production of the monoclonal antibody is from about 0.50 g/L to about 18.00 g/L, preferably from about 1.50 g/L to about 14.00 g/L and more preferably from about 2.00 g/L to about 12.50 g/L.

Typically, to achieve a relative Fab high mannose glycoform content of between about 2% and about 15% in the monoclonal antibody composition, the average concentration of glucose in the culture medium in the production phase of the culture process, in the production of the monoclonal antibody is between about 1.50 g/L and about 14.00 g/L.

As described herein, the production phase of a culture may be from 5 to about 18 days, for example about 10, 11, 12, 13, 14, 15, 16, 17 or 18 days as appropriate. In this disclosure harvest day is considered to be day 0 and the days of the production phase counted backwards therefrom. Thus, for a 14 day process the inoculation day would be day −14. As the end of the fermentation is the productive phase and is the time during which antibody is formed, counting from harvest results in more representative calculation for the average glucose concentration.

In this aspect, nutrient feeds comprising glucose and optionally other nutrients required for cell culture and growth and antibody expression are provided to the medium through the production phase in divided doses, via a continuous mechanism, or regular bolus feeds as described herein.

For the above methods, the average concentration of glucose takes into account the consumption of glucose by the cells.

In one aspect, the present invention envisages a method for regulating the relative content of high mannose Fab glycoforms of a glycosylated monoclonal antibody comprised in the composition of the invention, which method comprises, during the production phase of fermentation, optimising the concentration of glucose in a culture medium used for producing the glycosylated monoclonal antibody by fermentation therein of a eukaryotic cell expressing the monoclonal antibody. Under standard fermentation conditions, the concentration of other nutrients may also be optimised. The glucose concentration in the culture medium is optimised during all or a part of the production phase.

In a further aspect, the present invention provides a monoclonal antibody composition obtainable by a method set out above.

Glucose Supplementation

In the methods of the present invention, the concentration of glucose may be optimised, optionally by monitoring and controlling the concentration in the culture medium, over the whole or a part of the culture period, e.g. over the whole or a part of the production phase, so that the average concentration of glucose results in the desired glycoform distribution in the Fab portion of the expressed antibody.

With experience of appropriate concentrations of glucose required to achieve the desired high mannose glycoform distribution, monitoring of the concentrations of glucose may not be required. In this situation the inclusion in or addition of glucose and optional other components to the initial culture medium and/or in feeds over the whole or a part of the duration of the culture as required will achieve the desired glycoform distribution.

In one embodiment monitoring and control of glucose concentrations is performed during the production phase, typically from day −14 to day 0 (harvest), e.g. from day −13 to day 0, day −12 to day 0, day −11 to day 0, day −10 to day 0, day −9 to day 0, day −8 to day 0, day −7 to day 0, day −6 to day 0, day −5 to day 0 or day −4 to day 0 of the production phase.

In one embodiment, monitoring and control of glucose concentrations, is performed during the production phase, typically from day −14 to any one of days −1, −2, −3, −4 or −5, e.g. from day −14 to day −1, day −14 to day −2, day −14 to day −3, day −14 to day −4, day −14 to day −5; from day −13 to day −1, day −13 to day −2, day −13 to day −3, day −13 to day −3, day −13 to day −4, day −13 to day −5; from day −12 to day −1, day −12 to day −2, day −12 to day −3, day −12 to day −4, day −12 to day −5; from day −11 to day −1, day −11 to day −2, day −11 to day −3, day −11 to day −4, day −11 to day −5; from day −10 to day −1, day −10 to day −2, day −10 to day −3, day −10 to day −4, day −10 to day −5; from day −9 to day −1, day −9 to day −2, day −9 to day −3, day −9 to day −4, day −9 to day −5; from day −8 to day −1, day −8 to day −2, day −8 to day −3, day −8 to day −4, day −8 to day −5; from day −7 to day −1, day −7 to day −2, day −7 to day −3, day −7 to day −4, day −7 to day −5; from day −6 to day −1, day −6 to day −2, day −6 to day −3, day −6 to day −4, day −6 to day −5; from day −5 to day −1, day −5 to day −2, day −5 to day −3, day −5 to day −4; or from day −4 to day −1, day −4 to day −2 or day −4 to day −3 of the production phase.

In a preferred embodiment, the glucose concentration is monitored and controlled from day −7 to day 0 of the production phase.

In a preferred embodiment, the glucose concentration in the culture medium is optimised, optionally by monitoring and controlling the concentration from day −7 to day 0 of the production phase. The glucose concentration and optionally the concentration of other nutrients may be monitored daily, twice daily or more frequently if desired. Alternatively, monitoring may take place every 2 days, every 3 days, every 4 days, every 5 days, or once or twice during the production phase. When more than one nutrient is being monitored, they may all be monitored on the same or different days, at the same or different times.

Methods for monitoring the concentration of glucose are described below. Control of the glucose concentration in the culture medium is typically by addition of glucose to the medium. Regulating the concentration of glucose to achieve the desired average over the production phase is, optionally, by the inclusion of glucose in the culture medium, e.g. the base medium and/or by the addition thereof to the medium, e.g. by methods typical in the art and/or as described below. If desired, the concentration of other nutrients can be monitored at the same time, or a different time, to the monitoring of the glucose concentration and the concentration of those other nutrients adjusted as desire using methods typical in the art.

The present disclosure thus includes a method for regulating the relative content of high mannose Fab glycoforms of a glycosylated monoclonal antibody comprised in the composition of the invention, which method comprises adding glucose to a medium comprising a eukaryotic cell capable of expressing the glycosylated monoclonal antibody during the production phase of fermentation.

In this aspect, glucose may be added to the medium during all or a part of the production phase, typically on at least days −7 to day 0 thereof. As set out below, the amount of glucose added to the medium depends on the amount of glucose in the base medium, the consumption of glucose by the cells and the average glucose concentration over all or part of the production phase that correlates with the desired relative Fab high mannose content in the antibody produced. These features are set out in more detail below.

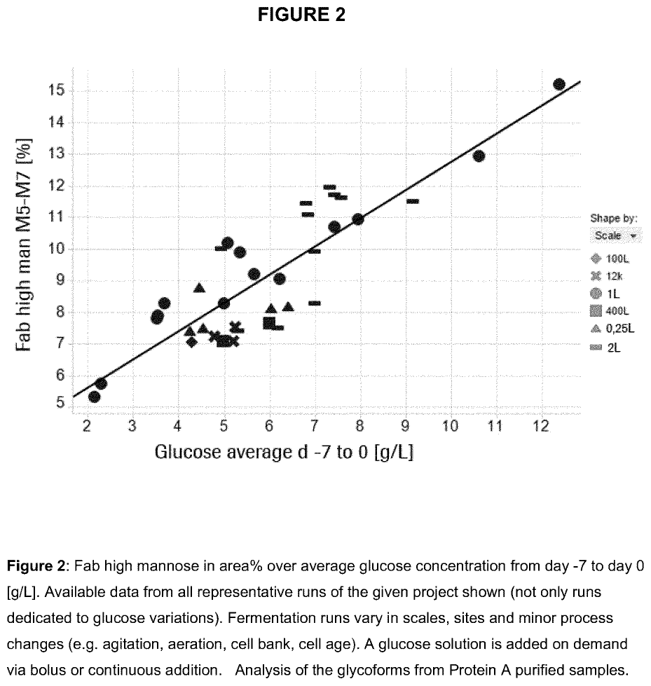

The present disclosure shows a correlation between the average glucose concentration in the culture medium over all or a part of the production phase and the relative Fab high mannose content in the antibody produced. Thus, in a preferred aspect, and as illustrated in FIG. 2:
if the desired relative content of high mannose Fab glycoforms (i.e. Man5, Man6 and Man7) of a glycosylated monoclonal antibody resulting from the fermentation is about 7%, the average concentration of glucose in the culture medium between about day −7 and harvest (day 0) may be between about 3.00 g/L and about 6.00 g/L;
if the desired relative content of high mannose Fab glycoforms (i.e. Man5, Man6 and Man7) of a glycosylated monoclonal antibody resulting from the fermentation is about 10.5%, the average concentration of glucose in the culture medium between about day −7 and harvest (day 0) may be between about 6.00 g/L and about 9.00 g/L;
if the desired relative content of high mannose Fab glycoforms (i.e. Man5, Man6 and Man7) of a glycosylated monoclonal antibody resulting from the fermentation is about 13%, the average concentration of glucose in the culture medium between about day −7 and harvest (day 0) may be between about 9.00 g/L and about 11.00 g/L; and
if the desired relative content of high mannose Fab glycoforms (i.e. Man5, Man6 and Man7) of a glycosylated monoclonal antibody resulting from the fermentation is about 15%, the average concentration of glucose in the culture medium between about day −7 and harvest (day 0) may be between about 11.00 g/L and about 14.00 g/L.

In an alternative aspect:
if the desired relative content of high mannose Fab glycoforms (i.e. Man5, Man6 and Man7) of a glycosylated monoclonal antibody resulting from the fermentation is about 0-6%, the average concentration of glucose in the culture medium between about day −7 and harvest (day 0) may be between about 0 g/L and about 3.00 g/L;
if the desired relative content of high mannose Fab glycoforms (i.e. Man5, Man6 and Man7) of a glycosylated monoclonal antibody resulting from the fermentation is about 6-8%, the average concentration of glucose in the culture medium between about day −7 and harvest (day 0) may be between about 3.00 g/L and about 6.00 g/L;
if the desired relative content of high mannose Fab glycoforms (i.e. Man5, Man6 and Man7) of a glycosylated monoclonal antibody resulting from the fermentation is about 8-10%, the average concentration of glucose in the culture medium between about day −7 and harvest (day 0) may be between about 4.00 g/L and about 8.00 g/L;
if the desired relative content of high mannose Fab glycoforms (i.e. Man5, Man6 and Man7) of a glycosylated monoclonal antibody resulting from the fermentation is about 10-12%, the average concentration of glucose in the culture medium between about day −7 and harvest (day 0) may be between about 6.00 g/L and about 10.00 g/L; and
if the desired relative content of high mannose Fab glycoforms (i.e. Man5, Man6 and Man7) of a glycosylated monoclonal antibody resulting from the fermentation is about 12-15%, the average concentration of glucose in the culture medium between about day −7 and harvest (day 0) may be between about 9.00 g/L and about 14.00 g/L.

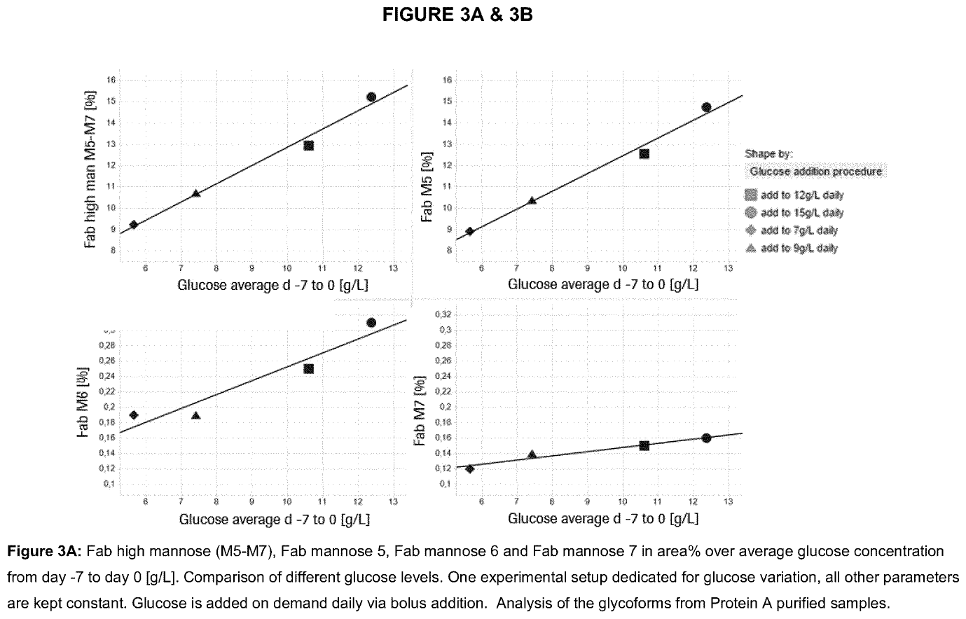

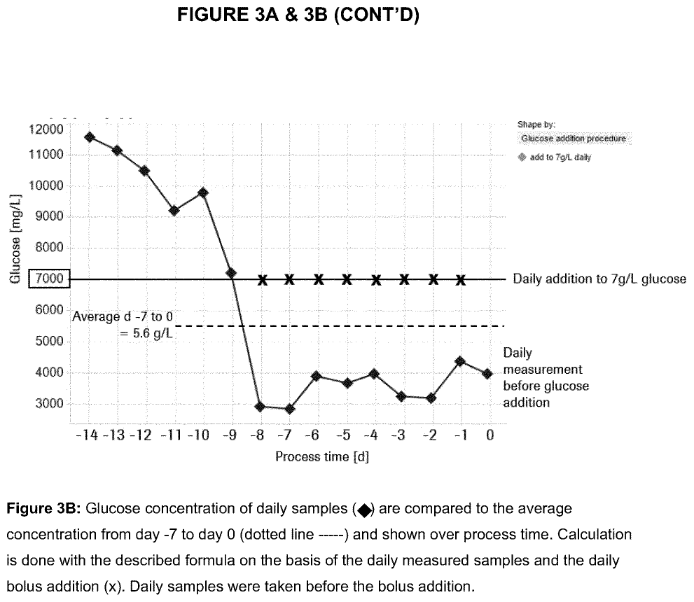

To achieve an average glucose concentration over all or a part of the production phase, the glucose concentration in the culture medium may be determined e.g. daily, and glucose added to the medium as necessary depending on the determined concentration. Glucose is consumed during culture. As illustrated in FIG. 3C, for a desired glucose average concentration over day −7 to day 0 of the production phase of 5.60 g/L, an amount of glucose is added daily to the culture after measurement of the glucose concentration in the medium to bring the daily concentration to 7.00 g/L. The amount of glucose added to the culture to achieve the desired average concentration thus depends on the measured concentration. A glucose concentration that is below the detection limit will be noted as 0 g/L.

The above relative Fab high mannose content and correlated average glucose concentrations are particularly applicable to a method for the production of Gantenerumab.

Glucose and other nutrients will typically be present in or added to the culture medium, i.e. the base medium into which the monoclonal antibody producing cells are transferred for the production phase. Transfer may be, for example, from a medium tailored for growth of the cells.

The precise nature of the base medium is not essential to the present invention. Chemically defined media have been extensively developed and published in recent history, including such media for culture of mammalian cells. All components of defined media are well characterized and such media do not contain complex additives such as serum and hydrolysates. Typically these media include defined quantities of purified growth factors, proteins, lipoproteins and other substances which may otherwise be provided by serum or extract supplement. Such media have been produced with the sole purpose of supporting highly productive cell cultures.

Certain defined media may be termed low protein media or may be protein free if the typical components of low protein media, insulin and transferrin, are not included. Serum free media may otherwise be used in the methods of the present invention. Such media normally do not contain serum or protein fractions, but may contain undefined components.

Examples of commercially available culture media include Ham's F10 (Sigma), Minimal Essential Medium (MEM, Sigma), RPMI-1640 (Sigma) and Dulbecco's Modified Eagle's Medium (DMEM, Sigma) and chemically defined media and feed supplements sold by Life Technologies. Any such media may be supplemented as necessary with hormones and/or other growth factors (such as insulin, transferrin or epidermal growth factor); salts (such as sodium chloride, calcium, magnesium and phosphate), amino acids, buffers (such as HEPES); nucleosides (such as adenosine and thymidine), antibiotics (such as GENTAMYCIN™), and glucose or an equivalent energy source. Any of these media may be used as the base medium, with addition of glucose and other nutrients as necessary to follow the methods described herein.

In a preferred embodiment, the cells are cultured in a chemically defined medium comprising glucose and other nutrients typically required for cell culture and growth and expression of antibody.

The necessary nutrients for the medium, including their concentrations for a particular cell line, are determined empirically and without undue experimentation as described in, for example, Mammalian Cell Culture, Mather (Plenum Press: NY 1984); Barnes and Sato, Cell 22 (1980) 649 or Mammalian Cell Biotechnology: A Practical Approach M. Butler (IRL Press, 1991). A suitable medium contains a basal medium component, such as DMEM/HAM F12-based formulation with modified concentrations of some components, such as amino acids, salts, sugar and vitamins, and optionally containing glycine, hypoxanthine, thymidine, recombinant human insulin, hydrolyzed peptone, such as PRIMATONE HS™ or PRIMATONE RL™ (Sheffield, England) or the equivalent, a cell protective agent, such as PLURONIC F68™ or the equivalent pluronic polyol and an antibiotic, such as GENTAMYCIN™.

Glucose can be added to the fermentation process as part of a nutrient feed, either as a bolus or continuous addition. Typically in the method of the present invention, three nutrient feeds will be given by bolus during the production phase. These bolus feeds may or may not contain glucose and other nutrients depending on the requirements of the cell and the production protocol being used. If, for example, glucose is a part of the nutrient feed, typically no separate addition of glucose is required on a day when a nutrient feed is given. If more than one bolus feed is given, each bolus feed may contain the same or different concentrations of glucose. The volume of a bolus or continuous feed is determined based on the needs of the culture. Methods for doing this are routine in the art. The nutrient feed may consist of the same medium into which the culture was inoculated, or may be formulated specifically for the particular culture. Typically such a nutrient feed will contain other components depleted from the cell culture medium for example by cell metabolism and required to ensure biomass generation and antibody production. Such supplementary components may include e.g. hormones, growth factors, ions, vitamins, nucleosides, trace elements, amino acids or lipids. Alternatively, glucose may be added to the fermentation process as a single nutrient. The supplementary components may be added in combination or singly, once or in a series of additions as required to replenish the depleted nutrients.

The frequency or volume or mode of addition of glucose to the cell culture medium when using the above average concentration method is not particularly important to the present invention, so long as an average concentration (to achieve the desired glycoform distribution) of glucose in the culture medium over the production phase is maintained.

Glucose may be added to the fermentation process by continuous addition, either in combination with or separately to other nutrients. When continuous feeding is used, the amount of glucose and/or those nutrients added to the medium in the production phase may be varied e.g. daily, every 2 days, every 3 days etc. or periodically throughout a 24 hour period, by adjusting the feed rate and/or volume, depending on the desired average concentration over the culture.

When added to the culture to affect high mannose Fab production according to the method of the invention, glucose may be added to the fermentation process on any or all days of the production (n) phase. The duration of the production phase may depend on the culture method used and/or may depend on the cell density used for inoculation of the medium, for example an inoculation cell density of about 1×10⁵ to about 20×10⁵ cells/mL will typically require a production phase of up to 10-18 days. However, when a higher inoculation cell density is used, e.g. from about 21×10⁵ to about 200×10⁵ cells/ml, the duration of the production phase may decrease to, for example 6 to 10 days. A high inoculation cell density can be reached with, for example, an intensified process, e.g. a perfusion process at the n-1 step of cultivation. In one embodiment of the present invention the production phase is up to 7-18 days from inoculation, preferably 7-10 days or 10-18 days or 14-17 days.

In this disclosure, harvest day is considered to be day 0 and therefore, for a 14 day production process, the days are counted backwards, such that the inoculation day would be day −14. As the end of the fermentation is the productive phase and is the time during which antibody is formed and expressed, counting from harvest results in a more representative calculation for the average glucose concentration. Thus calculation of the average glucose concentration between day −7 to day 0 is independent of the culture duration and the inoculation cell density of the production phase. In the present invention a calculation of the average from day 7 to 14 (where harvest is on day 14 and counting is “forwards”) equals the calculation of the average from day −7 to day 0, where harvest is on day 0 and counting is “backwards”.

In one aspect of the methods of the invention, glucose is added to the culture medium to achieve a desired average glucose concentration over the production phase or a part thereof, resulting in the desired relative percentage of high mannose glycoforms of the monoclonal antibody. The period of the production phase for which the average is calculated may depend on the cell density used for inoculation of the medium and thus the duration of the production phase. Thus, with a shorter production phase, the days over which the average is calculated/maintained is also shortened. For example with an inoculation cell density of about 1×10⁵ to about 20×10⁵ cells/ml and a production phase of 10-18 days, the average glucose concentration is typically calculated from any combination of one of days −18, −17, −16, −15, −14, −13, −12, −11, −10, −9, −8 or −7 with any one of days 0, −1, −2, −3, −4 or −5. For example, the average glucose concentration can be calculated over day −18 to day 0, day −18 to day −1, day −18 to day −2, day −18 to day −3, day −18 to day −4, day −18 to day −5; over day −17 to day 0, day −17 to day −1, day −17 to day −2, day −17 to day −3, day −17 to day −4, day −17 to day −5; over day −16 to day 0, day −16 to day −1, day −16 to day −2, day −16 to day −3, day −16 to day −4, day −16 to day −5; over day −15 to day 0, day −15 to day −1, day −15 to day −2, day −15 to day −3, day −15 to day −4, day −15 to day −5; over day −14 to day 0, day −14 to day −1, day −14 to day −2, day −14 to day −3, day −14 to day −4, day −14 to day −5; over day −13 today 0, day −13 to day −1, day −13 to day −2, day −13 to day −3, day −13 to day −3, day −13 to day −4, day −13 to day −5; over day −12 to day 0, day −12 today −1, day −12 to day −2, day −12 to day −3, day −12 to day −4, day −12 to day −5; over day −11 to day 0, day −11 to day −1, day −11 to day −2, day −11 to day −3, day −11 to day −4, day −11 to day −5; over day −10 to day 0, day −10 to day −1, day −10 to day −2, day −10 to day −3, day −10 to day −4, day −10 to day −5; over day − to day 0, day −9 to day −1, day −9 to day −2, day −9 to day −3, day −9 to day −4, day −9 to day −5; over day −8 to day 0, day −8 to day −1, day −8 to day −2, day −8 to day −3, day −8 to day −4, day −8 to day −5; over day −7 to day 0, day −7 to day −1, day −7 to day −2, day −7 to day −3, day −7 to day −4, day −7 to day −5 of the production phase.

If the inoculation cell density is greater than about 21×10⁵ cells/ml and up to about 200×10⁵ cells/ml, the production phase will typically be 6 to 10 days and the average glucose concentration is typically calculated from day −9 to day 0, day −8 to day 0 or day −7 to day 0, day −9 to day −1, day −8 to day −1 or day −7 to day −1, day −9 to day −2, day −8 to day −2 or day −7 to day −2, day −9 to day −3, day −8 to day −3 or day −7 to day −3 or day −9 to day −4, day −8 to day −4 or day −7 to day −4.

In a preferred embodiment, when the cells are cultured in a medium already containing glucose (i.e. the base medium for the production phase contains glucose) and supplemental glucose is added to the culture to affect high mannose Fab production according to the method of the invention, glucose may be added to the fermentation process on any one or all of days −18 to 0, days −17 to 0, days −16 to 0, days −15 to 0 or days −14 to 0, days −18 to −1, days −17 to −1, days −16 to −1, days −15 to −1 or days −14 to −1, days −18 to −2, days −17 to −2, days −16 to −2, days −15 to −2 or days −14 to −2, days −18 to −3, days −17 to −3, days −16 to −3, days −15 to −3 or days −14 to −3, days −18 to −4, days −17 to −4, days −16 to −4, days −15 to −4 or days −14 to −4 or days −18 to −5, days −17 to −5, days −16 to −5, days −15 to −5 or days −14 to −5 of the production phase, preferably on any one or more, or combination of, or all of, days −8 or −7 to day 0 of the production phase.

When the methods of the invention comprise optimising the glucose concentration in the culture medium over the period of the culture, typically over all or a part of the production phase, one or more other nutrients, e.g. amino acids required for cell growth and recombinant glycoprotein production may be present in the base medium and/or supplemental feeds, but the method then does not require optimisation of the concentration of those nutrients in the culture medium to achieve the desired glycoform distribution.

In a more preferred embodiment, glucose is supplemented “on demand”, i.e. to maintain the average concentration (typically from about day −7 of the production phase to harvest, day 0) in the culture medium, dependent on the amount of Fab high mannose required. In this alternative, addition of glucose “on demand” means that glucose is supplemented when the measured concentration of glucose in the culture medium is or falls below a particular level. In this aspect, the concentration of the glucose in the culture medium is monitored and controlled to achieve an average glucose concentration over all or a part of the production phase, which is correlated with the desired relative content of the Fab high mannose glycoform of the monoclonal antibody being produced. In one embodiment, a glucose containing nutrient feed is given at days −11, −8 and −5 of the production phase.

In one aspect, the cells are cultured wholly or for part of the process by perfusion cultivation, optionally together with a fed-batch process. Medium perfusion through the culture process allows the average glucose concentration to be maintained within the optimised ranges throughout the culture.

If monitoring of the glucose concentration is not required, e.g. because the rates of glucose consumption and addition are known from experience with earlier fermentation runs, the glucose may nonetheless be supplemented on demand or following a previously prescribed regimen.

The amount of glucose added to the fermentation (base) medium and in each supplemental/bolus feed, when more than one supplemental feed is used, may be the same or different. Typically the amount of glucose added to the fermentation medium in each supplemental/bolus feed will depend on the need of the cells and the measured concentration of those nutrients in the fermentation medium at that time.

In one embodiment, the disclosure provides a method for producing a relative content of less than 20%, e.g. about 15%, Fab high mannose glycoforms of a monoclonal antibody expressed from a eukaryotic cell cultured in a cell culture medium comprising glucose, which method comprises:
cultivating the eukaryotic cell in a cell culture medium comprising glucose;
measuring the glucose concentration in the cell culture medium daily during the production phase of the cell culture; and
adding glucose to the cell culture medium daily to achieve a glucose concentration of about 15 g/L after said addition.

In one embodiment herein, for example, to obtain a relative concentration of Fab N-linked high mannose glycoform of Gantenerumab (as described above) of about 9% or less, when the measured concentration of glucose in the culture medium is less than about 2.00 g/L, then about 5.00 g/L of glucose is added, or when the measured concentration of glucose in the culture medium is between about 2.00 g/L and about 5.50 g/L, then about 4.00 g/L of glucose is added to the culture medium. As set out above, a determination of the glucose concentration in the medium need not be made, and supplementation could follow a set plan based on previous experience.

As illustrated in FIGS. 2 and 3A there is a relationship between the average glucose concentration over day −7 to day 0 of the production phase and the % of N-linked high mannose Fab regions in the antibody (Gantenerumab) in the composition, relative to the total number of glycosylated Fab regions. Thus, as illustrated in FIG. 3A, to obtain about 9% high mannose Fab regions, glucose is added daily to the culture medium to achieve a glucose concentration in the medium after glucose addition of about 7.00 g/L, such that the average glucose concentration in the culture medium over day −7 to day 0 of the production phase is about 4.00 to 7.00 g/L. To obtain approximately about 10.5% high mannose Fab regions, glucose is added daily to the culture medium to achieve a glucose concentration in the medium after glucose addition of about 9.00 g/L such that the average glucose concentration in the culture medium over day −7 to day 0 of the production phase is about 6.00 to about 9.00 g/L. To obtain approximately about 13% high mannose Fab regions, glucose is added daily to the culture medium to achieve a glucose concentration of about 12.00 g/L in the culture medium after glucose addition such that the average glucose concentration in the culture medium over day −7 to day 0 of the production phase is about 9.00 to about 11.00 g/L. To obtain approximately about 15% high mannose Fab regions, glucose is added daily to the culture medium to achieve a glucose concentration of about 15 g/L in the medium after glucose addition such that the average glucose concentration in the culture medium over day −7 to day 0 of the production phase is about 11.00 to about 14.00 g/L.

Thus, to obtain a relative concentration of Fab N-linked high mannose glycoform of Gantenerumab (as described above) of about 3-10%, the average glucose concentration in the culture medium over day −7 to day 0 of the production phase may be from about 0 g/L to about 8.00 g/L.

In one aspect, the above average glucose concentrations are used to obtain a relative concentration of Fab N-linked high mannose glycoform of Gantenerumab of about 3-10%.

In a particularly preferred embodiment, the monoclonal antibody is Gantenerumab or a bi-specific antibody comprising Gantenerumab. In these embodiments, the monoclonal antibody has a V_{H} and a V_{L} CDR amino acid sequences as set out in SEQ ID Nos:1-6, above, a V_{H} and a V_{L} domain amino acid sequences of SEQ ID NO:7 and SEQ ID NO:8, or a heavy and light chain comprising the amino acid sequence of SEQ ID NO:9 and 10 and, to obtain a relative concentration of Fab N-linked high mannose glycoform of Gantenerumab of about 5-9%, in one aspect the average glucose concentration in the culture medium over day −7 to day 0 of the production phase may be from about 4.00 g/L to about 6.10 g/L, preferably from about 4.50 g/L to about 5.60 g/L and more preferably about 5.00 g/L.

Typically, when the measured concentration of glucose in the culture medium during the production phase is less than about 2.00 g/L then about 5.00 g/L of glucose is added to the culture medium, or when the measured concentration of glucose in the culture medium during the production phase is between about 2.00 g/L and about 5.50 g/L, then about 4.00 g/L of glucose is added to the culture medium, with glucose additions being from a stock solution (500 g/L). Measurements of glucose concentration in the medium and subsequent glucose addition when necessary can take place at the frequencies set out herein, with the aim of reducing or eliminating fluctuations in glucose concentration. Typically, with glucose addition as outlined herein, any glucose measurement of about 0 g/L will be addressed either by continuous or bolus addition, to prevent negative impact on the cells. As set out above, a determination of the glucose concentration in the medium need not be made, and supplementation would follow a set plan based on previous experience.

In an alternative embodiment, e.g. when culturing in a 12K (12,000 litre) fermenter, glucose is added to the culture medium if the measured concentration of glucose in the culture medium falls below about 4.00 g/L. This could be achieved by adding e.g. about 80 L from a stock solution containing 500 g/L glucose. Sampling to determine the glucose concentration may be performed once or twice daily.

Typically glucose is added as a 50% stock solution and formulae for the calculation of the amount of stock solution required are:

V_bolus [ml]=c_Glc [mg/L]*Vferm [L]/500 mg/ml Formula 1:

M_bolus [g]=V_bolus [ml]*1,224 g/ml Formula 2:

V_bolus is the volume of feed to be added as a bolus, c_Glc is the target concentration to be added to the fermenter, Vferm is the fermenter volume and M_bolus is the weight of the feed to be added as a bolus.

In one example, with a 1 L fermenter and glucose measured at 1.80 g/L, thus requiring about 5.00 g/L glucose to be added, the amount/volume of glucose/50% glucose stock solution added can be calculated as:

V_bolus [ml]=5000 mg/L*1 L/500 mg/mL=10 mL Glucose

M_bolus [g]=10 ml*1,224 g/ml=12,24 q Glucose.

If glucose is added to the culture medium as part of a standard feeding process during fed-batch production of the monoclonal antibody in question, then supplemental feeding may or may not additionally be required depending on the concentration of glucose included in the culture medium and/or the average glucose concentration measured over the production phase. For example, on days when a standard bolus feed is provided, the glucose portion of that bolus feed would typically provide sufficient glucose to ensure that the average glucose concentration over culture to harvest remains at the desired concentration, depending on the % high mannose glycoform desired, as set out above.

The method of the present invention also involves monitoring the average glucose concentration in the culture medium. Typically, the glucose concentration in the culture medium is monitored by measurement. In a preferred embodiment, the glucose concentration in the culture medium is measured daily, every two days, every three days etc, or twice a day, or three times a day, etc., before a determination of the necessity for supplementation is made. Most preferably the glucose concentration is measured daily or twice daily. It is not important whether the measurement is taken at the same time(s) every 24 hours, or at a different time(s) during each 24-hour period. Most preferably, the measurement is taken once a day before adding a bolus feed (nutrient feed, glucose feed, etc). Monitoring may be only of the glucose concentration in the culture medium i.e., monitoring of the concentration of other nutrients may not be required, or vice versa. Supplementation with glucose and optionally other nutrients takes place after measurement of the respective nutrient concentration in the culture medium if required.

In one embodiment, calculation of the average glucose concentration in the culture process may be performed using the equation:
(i) for continuous glucose addition and for bolus addition with sample taken before and after the bolus addition:

$$c ( glucose avg ) = \frac{{\sum }_{i = 1}^{n} {a}_{i}}{n}$$

where e.g., n=number of samples of cultivation (samples from day −7 to day 0 taken into account), i is the index of summation (indexing number of samples), and aᵢ is the measured concentration of glucose in the culture medium (e.g. in g/L) from sample i to n (for day −7 to day 0). No glucose containing bolus nutrient feed is added. Typically, one sample per day is taken.
(ii) for bolus glucose addition without taking a sample after bolus addition:

$$c ( glucose avg ) = \frac{{\sum }_{i = 1}^{n} {a}_{i} + {\sum }_{k = 1}^{m} ( {a}_{k} + {b}_{k} + {f}_{k} )}{n + m}$$

where e.g., n=number of samples of cultivation (samples from day −7 to day 0 taken into account), m is the number of glucose additions via glucose or nutrient feed (additions from day −7 to day 0 taken into account), i is the index of summation indexing number of samples, k is the index of summation indexing the number of glucose additions, a; is the measured concentration of glucose in the culture medium (e.g. in g/L) from sample i to n (for day −7 to day 0), aₖ is the measured concentration of glucose in the culture medium (e.g. in g/L) that is taken shortly before a glucose bolus addition from glucose addition k to m (for day −7 to day 0), bₖ is the glucose addition (bolus) to the culture medium (e.g. in g/L based on the fermenter volume at the day of addition) from addition k to n (for day −7 to day 0), fₖ is the glucose addition via the nutrient feed addition (bolus) to the culture medium (e.g. in g/L based on the fermenter volume at the day of addition) from addition k to m (for day −7 to day 0). Typically one sample per day is taken, before addition of a nutrient feed or glucose bolus.

Thus, in the methods of the invention, the glucose concentration in the culture medium is measured and, depending on the measured concentration, the concentration of glucose in the culture medium is adjusted in order to achieve the average glucose concentration over the production phase. The amount of adjustment depends on the average glucose concentration over the production phase, which is determined according to the relative amount of Fab high mannose glycoform desired in the antibody composition.

Measurement of the glucose concentration may be off-line, i.e. take place in a sample of the culture medium or may be on-line or in situ, i.e. directly in the culture. Methods for the measurement of glucose concentration off-line are familiar to person of skill in the art, and may comprise use of Cedex Bio HT. A sample of cell culture fluid is centrifuged to separate cells and then analysed in the Bio HT. The Bio HT assay works as follows: glucose is phosphorylated by ATP in the presence of hexokinase (HK) to produce glucose-6-phosphate (G-6-P), which is oxidised by NADH in the presence of glucose-6-phosphate dehydrogenase (G-6-PDH). The rate of NADPH formation is measured UV-photometrically and is directly proportional to the glucose concentration.

When measured on-line/in situ, glucose concentration can be determined using a probe and analysis system, allowing on-line monitoring. An example of such a system is the BioPat® Trace (Sartorius) technology.

Spectroscopic methods, such as Raman, may otherwise be used to measure glucose concentrations, or an estimation of glucose concentration/consumption may be made based on e.g. oxygen consumption.

The concentration of any other nutrients in the culture medium can, if required, also be determined either in a sample removed from the medium or directly in the medium itself. Typically concentrations of, for example, amino acids are determined in samples of the medium, which may be analysed using e.g. the Thermo Scientific Dionex UltiMate 3000 Rapid Separation LC system or by Raman.

In the methods of the invention, the culture medium is supplemented with glucose when the measured glucose concentration is between certain limits, depending on the average glucose concentration required to achieve the desired relative amount of Fab high mannose glycoform in the composition.

In the alternative, a daily glucose addition procedure can be adopted, with the amount of glucose added to the culture (in g/L) being dependent on the desired % high mannose Fab relative to total glycosylated Fab required and the average glucose concentration over day −7 to day 0 of the production process.

Thus in one example:
(a) glucose is added daily to the culture medium during the production phase to achieve an average concentration over day −7 to day 0 of the production phase of about 3.00 to 6.00 g/L, to obtain approximately 7% high mannose Fab regions;
(b) glucose is added daily to the culture medium during the production phase to achieve an average concentration over day −7 to day 0 of the production phase of about 4.00 to 7.00 g/L, to obtain approximately 9% high mannose Fab regions;
(c) glucose is added daily to the culture medium during the production phase to achieve an average concentration over day −7 day 0 of the production phase of about 6.00 to 9.00 g/L to obtain approximately 10.5% high mannose Fab regions;
(d) glucose is added daily to the culture medium during the production phase to achieve an average concentration over day −7 to day 0 of the production phase of about 9.00 to 11.00 g/L to obtain approximately 13% high mannose Fab regions; and
(e) glucose is added daily to the culture medium during the production phase to achieve an average concentration over day −7 to day 0 of the production phase of about 11.00 to 14.00 g/L to obtain approximately 15% high mannose Fab regions.

Although the above requires daily addition of glucose, each daily supplement can be added in one or more doses, or can be via continuous addition.

Typically glucose is added in solution to the culture medium. This can be a stock solution or nutrient feed. The concentration of nutrient in the solution may vary, depending e.g. on the volume to be added, and vice versa.

In one aspect, in order to achieve less than about 20% N-linked high mannose Gantenerumab, Table 1A shows the observed average concentrations of glucose, taken over days −7 to 0 of the production phase (i.e. taking into account glucose present in base medium, feed medium and bolus additions).

TABLE 1A
Observed Averages

| 0-20% Fab | Days | Observed | Observed | |
|---|---|---|---|---|
| high man | Averaged | Minimum Value | Maximum Value | Unit |
| Glucose (Glc) | −7 to 0 | 2.14 | 12.37 | g/L |

In all of the above methods, it is most preferred that the antibody expressed by the cells in culture is an anti-human Abeta antibody, such as Gantenerumab.

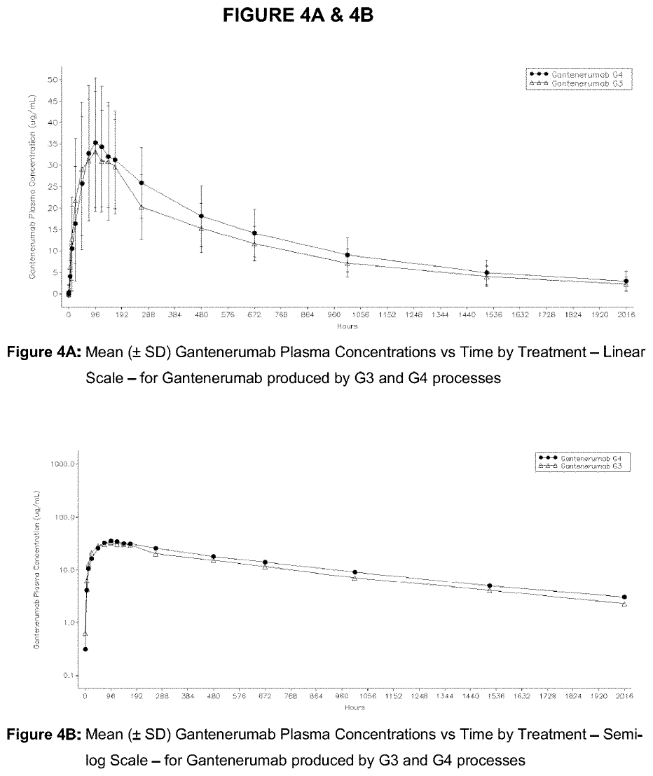

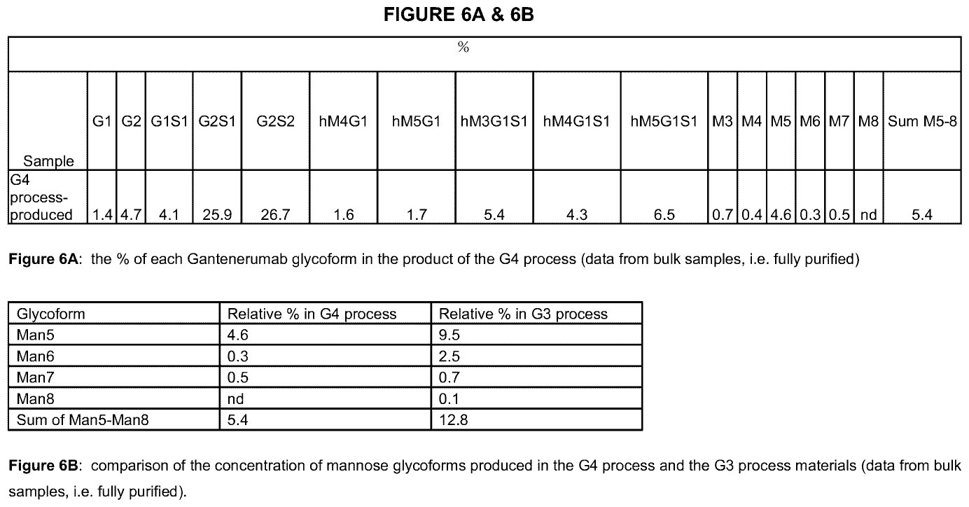

Our results have shown:
1. (see e.g. FIGS. 2 and 3A) that there is a strong correlation between the average glucose concentration over day −7 day 0 of the production phase and the relative content of Fab high mannose glycoforms of the monoclonal antibody. These effects can be transferred from small to large scale bioreactors.
2. (see e.g. FIGS. 4 & 5) that the production of a homogeneous population of antibodies in which the relative amount of a specific glycoform at the Fab N-glycosylation site is controlled results in a therapeutic with a consistent pharmacological profile.
3. An increase in bioavailability of monoclonal antibody of about 18% can be achieved using Fab high mannose glycoforms comprising between about 5 and 6% Man5-7 prepared according to the methods herein, compared to a high mannose glycoform comprising twice as much Man5-7 and prepared according to a different method (see FIG. 6B).

Cells, Production Media, Methods Etc

According to the methods of the present invention, the glycosylated monoclonal antibody is produced in a eukaryotic cell. Any eukaryotic cell susceptible to cell culture and to expression of glycosylated monoclonal antibodies may be used in accordance with the present invention. Typically, the eukaryotic cell is glucose-responsive. The eukaryotic cell is preferably a eukaryotic cell line which is capable of growth and survival when placed in suspension culture in a medium containing the appropriate nutrients and growth factors and which is typically capable of expressing and secreting large quantities of a particular glycosylated monoclonal antibody of interest into the culture medium.

In a preferred embodiment, the eukaryotic cell is a mammalian cell, a yeast cell or an insect cell.

When the eukaryotic cell is a mammalian cell, this may be, for example, an NSO murine myeloma cell line, a monkey kidney CVI line transformed by SV40 (COS-7, ATCC® CRL 1651); human embryonic kidney line 293S (Graham et al., J. Gen. Virol. 36 (1977) 59); baby hamster kidney cells (BHK, ATCC® CCL 10); mouse sertoli cells (TM4, Mather, Biol. Reprod. 23 (1980) 243); monkey kidney cells (CVI-76, ATCC® CCL 70); African green monkey kidney cells (VERO-76, ATCC® CRL 1587); human cervical carcinoma cells (HELA, ATCC® CCL 2): canine kidney cells (MDCK, ATCC® CCL 34); buffalo rat liver cells (BRL 3A, ATCC® CRL 1442); human lung cells (W138, ATCC® CCL 75): human liver cells (Hep G2, HB 8065); mouse mammary tumour cells (MMT 060562, ATCC® CCL 51); rat hepatoma cells (HTC, MI.54, Baumann et al., J. Cell Biol., 85 (1980) 1); and TR-1 cells (Mather et al., Annals N.Y. Acad. Sci. 383 (1982) 44), the PER.C6 cell line (Percivia LLC) and hybridoma cell lines.

Chinese Hamster Ovary cells (CHO, Urlaub and Chasin P.N.A.S. 77 (1980) 4216) or PER.C6 are preferred cell lines for practicing this invention. Known CHO derivatives suitable for use herein include, for example CHO/−DHFR (Urlab & Chasin, supra), CHOK1SV (Lonza), CHO-K1 DUC B11 (Simonsen and Levinson P.N.A.S. 80 (1983) 2495-2499) and DP12 CHO cells (EP 307,247).

When the eukaryotic cell is a yeast cell, this may be, for example, Saccharomyces cerevisiae or Pichia pastoris.

When the eukaryotic cell is an insect cell this may be, for example, Sf-9.

Chinese Hamster Ovary cells (CHO) and mouse myeloma cells (NSO, SP2/0) have become the gold-standard mammalian host cells for the production of therapeutic antibodies and most of these cell lines have been adapted to grow in suspension culture and are well-suited for reactor culture, scale-up and large volume production, with a productivity ranging from 1 to 8 g/L.

It is most preferred that in the present invention, the cell is a CHO cell, e.g. a CHOK1 cell.

The eukaryotic cell used in the present invention is selected or manipulated to produce recombinant glycosylated monoclonal antibody. Manipulation includes one or more genetic modifications such as introduction of one or more heterologous genes encoding the monoclonal antibody to be expressed. The heterologous gene may encode a monoclonal antibody either that is normally expressed in that cell or that is foreign to the host cell. Manipulation may additionally or alternatively be to up- or down-regulate one or more endogenous genes. Often, cells are manipulated to produce monoclonal antibody by, for example, introduction of a gene encoding the antibody and/or by introduction of control elements that regulate expression of the gene encoding the antibody. Genes encoding monoclonal antibody and/or control elements may be introduced into the host cell via vectors, such as a plasmid, phage or viral vector. Certain vectors are capable or autonomous replication in a host cell into which they are introduced whilst other vectors can be integrated into the genome of a host cell and are thereby replicated along with the host genome. Various vectors are publicly available and the precise nature of the vectors is not essential to the present invention. Typically vector components include one or more of a signal sequence, an origin of replication, one or more marker genes, a promoter and a transcription termination sequence. Such components are as described in WO 97/25428.

In a preferred aspect, the glycosylated monoclonal antibody produced according to the methods of the invention is Gantenerumab, as described above.

Biomass generation and glycoprotein expression from eukaryotic cells is achieved according to the method of the invention by culture of the cells under fermentation conditions. Any fermentation cell culture method or system that is amenable to the growth of the cells for biomass generation and expression of monoclonal antibody may be used with the present invention. For example, the cells may be grown in batch or fed-batch or perfusion cultures, where the culture is terminated after sufficient expression of the monoclonal antibody has occurred, after which the glycoprotein is harvested and, if required, purified. If a fed-batch culture is used, feeding of the culture may take place continuously, or periodically during culture. When multiple feeds are given, these may be given daily, every other day, every two days etc., more than once per day, or less than once per day, and so on, with the same or different feeding solutions for each feed. In a preferred embodiment, the cell culture method used in the present invention is fed-batch.

Reactors, temperatures and other conditions for fermentation culture of cells for biomass generation and the production of glycoproteins, such as oxygen concentration, carbon dioxide and pH, agitation, temperature and humidity are known in the art. Differing reactor volumes may be used through the fermentation process. For example, the cell culture is established by inoculating either shake flasks or a 20 L bioreactor and cultivating for about 21 days. After that, cells may be transferred to an 80 L bioreactor for about 3 days, a 400 L reactor for about 3 days and a 2,000 L reactor for about 2 days (stage n-1). The main fermentation, for production of antibody (n phase), takes places in, for example, a 12,000 L bioreactor.

Any conditions appropriate for culture of the selected eukaryotic cell can be chosen using information available in the art. The culture conditions, such as temperature, pH and the like, are typically those previously used with the host cell selected for expression and will be apparent to the person skilled in the art. If desired, the temperature and/or the pH and/or CO₂ could be altered during cultivation in order to increase yield and/or increase the relative amount of the desired monoclonal antibody quality.

The present invention provides cell culture under fermentation culture conditions. This is typically a multi-step culture procedure where the cells are cultivated in a number of steps or phases. According to this preferred procedure, the fermentation culture process, e.g. from frozen vials of cells, typically covers three distinct phases, i.e.:
i) the seed train, for recovery of the cells after the stress of thawing and to normalize cell doubling times, which can last between 14 and e.g. more than 60 days, depending on the speed of cell recovery and the scale of production. This phase can take place in shake flasks or in a bioreactor, for example a 20 L bioreactor.
ii) the growth phase, or inoculation train, comprising n-x phases, wherein x is typically 1 to 5, preferably 1 or 2 or 1, 2 or 3. These phases may also be referred to as a growth phase(s) wherein cells are inoculated into a medium suitable for promoting growth and biomass generation. Thus, the n-x phases are typically for the expansion of the culture for larger cultivation formats and the wash-out of the selected compound. When the n-x phases consist of an 3 phases, n-1, n-2 and n-3, each of the phases takes e.g. from 2 to 8 days, typically each lasting 2, 3 or 4 days; and
iii) the production phase, or the production of the recombinant glycoprotein in appropriate quantity and/or quality. The duration of this phase may depend on, for example, the nature of the recombinant cell as well as the quantity and/or quality of the expressed glycoprotein. Typically this phase will last between about 11 and about 20 days. Typically this main fermentation phase will take place in a 12,000 L bioreactor. Protein and/or cells may be harvested during and/or at the end of the production phase. In this disclosure, harvest is typically nominated as day 0.

Typically, with production in a 12,000 L bioreactor, the cells at harvest will be from 48-62 days old.

A transition phase may also be incorporated, being a period of time between the growth phase and the production phase. Generally a transition phase is the time during which culture conditions may be controlled to shift from growth to production. Various cell culture parameters that may be controlled include temperature, osmolality, vitamins, amino acids, sugars, peptones, ammonium and salts.

The cells may be maintained in the seed train or in the growth phase for a suitable period of time by, e.g. the addition of fresh medium or nutrient supplementation to existing medium as appropriate.

Any or all of the seed train, the growth phase and production phase may be continuous, or the cells from one phase may be used to inoculate the next phase, e.g. in a fresh medium.

In one aspect of the method of the present invention, the expressed monoclonal antibody is recovered from the cell culture supernatant. Recovery of the expressed monoclonal antibody either during or at the end of a culture period, preferably the production phase, can be achieved using methods known in the art. The expressed monoclonal antibody may be isolated and/or purified, e.g. from the cell culture supernatant, as necessary using techniques known in the art, such as protein A columns, ion exchange column purification and/or size exclusion column purification. The glycosylation profile of the monoclonal antibody prepared by the methods of the invention can be analysed using methods well known to those skilled in the art and described above, or, for example by removal and derivatization of N-glycans followed by e.g. normal phase (NP) HPLC analysis, weak cation exchange chromatography (WCX), capillary isoelectric focussing (cIEF), size-exclusion chromatography, POROS™ A HPLC Assay, Host cell Protein ELISA, DNA assay and western blot analysis. Such purification steps do not affect the glycoform content of the expressed antibody.

In a further aspect, the monoclonal antibody composition of the present disclosure is that directly resulting from the fermentation, i.e. is the culture supernatant.

3. Methods of Treatment/Therapeutic and Diagnostic Use

The compositions provided herein are particularly useful as pharmaceutical or diagnostic compositions. Such compositions typically comprise a pharmaceutically acceptable carrier.

The therapeutic or diagnostic utility of the glycosylated monoclonal antibody remains unchanged in the composition of the invention. Thus, a composition comprising a monoclonal antibody having N-glycosylation in the Fab region(s) thereof, wherein relative to the total amount of Fab glycosylated antibody in the composition, about 20% or less of monoclonal antibodies in the composition have an N-linked high mannose glycan in the Fab region(s) thereof of the invention can be used to treat any disorder in a subject for which the antibody comprised in the composition is appropriate.

Thus, for example, Gantenerumab is known as a diagnostic reagent in the detection of genuine human amyloid plaques in brain sections of Alzheimer's Disease patients and also as a therapeutic in the prevention or treatment of a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral hemorrhage, with amyloidosis Dutch type, Down's syndrome and neuronal disorders related to aging, and in a composition of the invention that utility will remain the same.

Therefore, the monoclonal antibody may be Gantenerumab. Accordingly, the present invention provides in one embodiment a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease, comprising administering to the individual a monoclonal antibody comprising a V_{H} CDR1 comprising the amino acid sequence of SEQ ID NO:1; a V_{H} CDR2 comprising the amino acid sequence of SEQ ID NO:2; a V_{H} CDR3 comprising the amino acid sequence of SEQ ID NO:3; a V_{L} CDR1 comprising the amino acid sequence of SEQ ID NO:4; a V_{L} CDR2 comprising the amino acid sequence of SEQ ID NO:5; and a V_{L} CDR3 comprising the amino acid sequence of SEQ ID NO:6, said composition comprising about 20% or less of a high mannose glycoform of said antibody relative to the total amount of V_{H} glycosylated antibody in the composition, wherein said glycosylation is N-glycosylation at Asn52 in the CDR2 of the antibody.

In a preferred aspect of this method, said composition comprises about 15% or about 10% or less of an N-linked high mannose Fab glycoform of said antibody relative to the total amount of V_{H} glycosylated antibody in the composition.

In an alternative embodiment, the present invention provides a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease, comprising administering to the individual a composition comprising a monoclonal antibody comprising a V_{H} domain comprising the amino acid sequence of SEQ ID NO:7; and a V_{L} domain comprising the amino acid sequence of SEQ ID NO:8; said composition comprising about 20% or less of a high mannose glycoform of said antibody relative to the total amount of V_{H} glycosylated antibody in the composition, wherein said glycosylation is N-glycosylation at Asn52 in SEQ ID NO:7.

In a preferred aspect of this method, said composition comprises about 15% or about 10% or less of a high mannose glycoform of said antibody relative to the total amount of V_{H} glycosylated antibody in the composition.

In an alternative embodiment, the present invention provides a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease, comprising administering to the individual a composition comprising a monoclonal antibody comprising a heavy chain comprising the amino acid sequence of SEQ ID NO:9; and a light chain comprising the amino acid sequence of SEQ ID NO:10; said composition comprising about 20% or less of a high mannose glycoform of said antibody relative to the total amount of V_{H} glycosylated antibody in the composition, wherein said glycosylation is N-glycosylation at Asn52 in SEQ ID NO:9.

In a preferred aspect of this method, said composition comprises about 15% or about 10% or less of a high mannose glycoform of said antibody relative to the total amount of V_{H} glycosylated antibody in the composition.

In the alternative, the present invention provides a composition comprising a monoclonal antibody comprising a V_{H} CDR1 comprising the amino acid sequence of SEQ ID NO:1; a V_{H} CDR2 comprising the amino acid sequence of SEQ ID NO:2; a V_{H} CDR3 comprising the amino acid sequence of SEQ ID NO:3; a V_{L} CDR1 comprising the amino acid sequence of SEQ ID NO:4; a V_{L} CDR2 comprising the amino acid sequence of SEQ ID NO:5; and a V_{L} CDR3 comprising the amino acid sequence of SEQ ID NO:6, said composition comprising about 20% or less of a high mannose glycoform of said antibody relative to the total amount of V_{H} glycosylated antibody in the composition, wherein said glycosylation is N-glycosylation at Asn52 in the CDR2 of the antibody, for use in a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease.

In a preferred aspect of this method, said composition comprises about 15% or about 10% or less of a high mannose glycoform of said antibody relative to the total amount of V_{H} glycosylated antibody in the composition.

In the alternative, the present invention provides a composition comprising a monoclonal antibody comprising a V_{H} domain comprising the amino acid sequence of SEQ ID NO:7; and a V_{L} domain comprising the amino acid sequence of SEQ ID NO:8; said composition comprising about 20% or less of a high mannose glycoform of said antibody relative to the total amount of V_{H} glycosylated antibody in the composition, wherein said glycosylation is N-glycosylation at Asn52 in SEQ ID NO:7, for use in a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease.

In a preferred aspect of this method, said composition comprises about 15% or about 10% or less of a high mannose glycoform of said antibody relative to the total amount of V_{H} glycosylated antibody in the composition.

In the alternative, the present invention provides a composition comprising a monoclonal antibody comprising a heavy chain comprising the amino acid sequence of SEQ ID NO:9; and a light chain comprising the amino acid sequence of SEQ ID NO:10; said composition comprising about 20% or less of a high mannose glycoform of said antibody relative to the total amount of V_{H} glycosylated antibody in the composition, wherein said glycosylation is N-glycosylation at Asn52 in SEQ ID NO:9, for use in a method of treating an individual with a disease associated with amyloidogenesis and/or plaque formation, such as dementia, Alzheimer's Disease, motor neuropathy, Parkinson's Disease, Amylotrophic Lateral Sclerosis (ALS), scrapie, HIV-related dementia and Creutzfeld-Jakob disease, hereditary cerebral haemorrhage, with amyloidosis Dutch type, Down's syndrome and neuronal disorders related to aging, preferably Alzheimer's Disease.

In a preferred aspect of this method, said composition comprises about 15% or about 10% or less of a high mannose glycoform of said antibody relative to the total amount of V_{H} glycosylated antibody in the composition.

In any, or all, of the above aspects, the monoclonal antibody Gantenerumab may also be N-glycosylated in the Fc region thereof.

Typically the individual is a human. A diagnosis of Alzheimer's Disease is based on the National Institute of Neurological and Communicative Disorders and Stroke/Alzheimer's Disease and Related Disorders Association (NINCDS/ADRDA) criteria for this diagnosis.

The compositions according to the present invention can be administered by a variety of methods known in the art. Exemplary route/modes of administration include subcutaneous injection, intravenous injection or infusion. In certain aspects the composition may be orally administered. As will be appreciated by the person skilled in the art, the route/mode of administration will vary depending on the desired results.

Co-therapy of the compositions of the present invention is also envisaged. Thus, in the case of Alzheimer's Disease, co-therapy with approved medicaments such as memantine, doneprezil, rivastigmine or galantamine is envisaged.

Dosage forms and regimens may be adjusted to provide the optimum desired response and may vary with the type and severity of the condition to be treated. Furthermore, for any particular subject, specific dosage regimens may be adjusted over time, according to the individual need and the professional judgement of the person administering or supervising the administration of the composition. Dosage forms and regimens do not, per se, form part of the present invention.

In a further aspect, the present invention provides a method for decreasing the clearance of a composition comprising a monoclonal antibody or parts or fragments thereof, the method comprising regulating the relative content of high mannose Fab glycoforms in the composition.

Typically, the present inventors have discovered that decreasing the relative content of high mannose Fab glycoforms in the antibody composition results in an increase in the AUC in humans. Thus, according to this aspect, a method for increasing the AUC by at least about 5 percentage points, for example an increase from 6 to 11 percentage points), of a composition comprising a monoclonal antibody or parts or fragments thereof, is provided by regulating the relative content of high mannose Fab glycoforms in the composition.

Methods for regulating the relative content of high mannose Fab glycoforms in the antibody composition are as described hereinabove.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### EXAMPLES

The following examples, including the experiments conducted and the results achieved, are provided for illustrative purposes only and should not be construed to include all possible embodiments along with the full scope of equivalents to which such claims are entitled.

Materials and Chemical Substances

Cell Lines

In the study described below we used a recombinant CHO-K1 cell line producing Gantenerumab. The resulting antibody is glycosylated in both the Fc and the Fab regions. The cell line was cultivated in a fed batch mode using proprietary chemically defined protein-free medium.

In Process Control—Cell Growth and Analyses of Metabolites

Cell growth and viability were analyzed by using an automated CedexHiRes device (Roche Innovatis, Bielefeld, Germany). For quantification of the product titer and the metabolites glucose, lactate and ammonium, cell culture fluid was centrifuged to separate cells and analyzed using a Cedex Bio HT system (Roche, Mannheim, Germany). The test principle for the glucose Bio HT assay works as follows: Glucose is phosphorylated by ATP in presence of hexokinase (HK) to glucose-6-phosphate (G-6-P), which is oxidized by NADH in presence of glucose-6-phosphate dehydrogenase (G-6-PDH). The rate of NADPH formation is measured UV-photometrically and is directly proportional to the glucose concentration.

In Process Control—Amino Acids

Free amino acid concentrations were detected in cell-free cultivation supernatant using an UltiMate 3000 RSLC (Rapid Separation (RS) HPLC).

Glyco Species Analysis

Initially harvested cell culture fluid has to be centrifuged to separate the cells and get a cell free supernatant. To separate the main impurities before analysis of certain product quality attributes, a Protein A step was performed:

Fc-containing antibodies or antibody-related molecules are Protein A affinity purified (e.g. MabSelect SuRe, GE Healthcare) using a Tecan Freedom EVO® liquid handling workstation, deck size 150 cm, and Atoll MediaScout® RoboColumn technology in 96 array format. A slow pipetting speed of ≤5 μl/s is applied in all steps. Briefly, RoboColumns (column volume (CV) 200 μl, inner dimensions 10 mm bed height and 5 mm inner diameter) are pre-cleaned with 2 CV of Regeneration Buffer (0.2 M NaOH). After 5 minutes of incubation, RoboColumns are conditioned with 10 CV Equilibration Buffer (0.025 M NaCl, 0.025 M Tris, pH 7.2). The conditioned RoboColumns are loaded with max. 4 mg protein per column (the load-volume of Harvested Cell Culture Fluid (HCCF) is adjusted accordingly). After washing with 4 CV Equilibration Buffer, bound protein is eluted with 4 CV Elution Buffer (0.05 M Acetate, pH 3.7). The pH of the eluates is neutralized immediately by addition of 1 M Tris, pH 11. Absorbance at 280 nm is measured using a Tecan Infinite M200 plate reader. RoboColumns are rinsed with 3 CV Equilibration Buffer before they are regenerated with 2 CV of Regeneration Buffer followed by an incubation of 10 minutes. Finally, RoboColumns are flushed with 5 CV Equilibration Buffer and 4 CV of 20% Ethanol for storage at 4° C.

Samples may otherwise, or in addition, be fully purified (bulk samples). The method for purification of the antibody or parts thereof is not expected to have any significant effect on the % glycoform obtained, but may depend on the number of purification steps used. In examples 1 and 2 and FIGS. 2-3B herein the glycoform % are determined from a Protein A purified fraction. The values mentioned in examples 3 and 4 and FIGS. 4A to 8B are obtained from fully purified samples (bulk samples). The purification was performed using several purification steps.

The analysis of the relative distribution of the N-glycans implemented for the herein used antibody can be described as follows: in the first step, Fc glycans are cleaved from the antibody backbone by endoglycosidase PNGaseF. Released Fc glycans are separated from the protein via ultrafiltration and are collected. After protein sample rebuffering, Fab glycans are released by “rapid PNGaseF” digestion and then separated from the protein via ultrafiltration. Subsequently, Fc and Fab glycans are separately labelled with 2-AB (2-Aminobenzamide) and excessive label is removed. Finally, Fc and Fab glycans are independently analyzed by HILIC (hydrophilic interaction chromatography)-UHPLC (Ultra high performance liquid chromatography) with fluorescence detection.

The percentage of each glycoform (i.e. high mannose or other glycoform) in the preparation may be calculated e.g. from a chromatogram of the glycans produced. FIG. 9 illustrates such a chromatogram. To calculate the percentage of M5-M7 glycoforms, a baseline is drawn, e.g. in FIG. 9 from 5 to 39 minutes and then the area under the curve with that baseline is calculated (A1). The areas of the peaks M5-M7 is then divided by A1 and multiplied by 100 to give the percent M5-M7 to give the percentage figure (%) or area %.

### Example 1

Regulation of Fab Glycosylation by Varying the Glucose Addition

In FIG. 2 all data from representative runs is shown. The data are collected from fermentation runs in different scales (0.25 L, 2 L, 100 L, 400 L, and 12,000 L). The fermentations are conducted for different purposes, such as optimisation of the fermentation process and material supply, and are not dedicated especially to the evaluation of the glucose influence. Apart from minor variations that can be ignored according to process experience (e.g. agitation, aeration, cell bank, cell age), the parameters are the same for all runs apart from the glucose addition.

Glucose was added in different ways to vary the overall amount of glucose added to the process. When a continuous addition of a glucose solution is employed, addition is implemented via a pump and scale. The addition rate is adjusted daily depending on the measurement of glucose in the medium—measurement may be either external or may take place directly in the medium. Methods for direct measurement, i.e. in the medium itself, include the use of a glucose probe measuring the glucose level in the culture internally or are based on the oxygen consumption of the cells via off gas analytics.

Alternatively, glucose is added as bolus addition based on an external measurement using, e.g. the Cedex Bio HT apparatus. A certain volume of a 50% glucose solution (500 g/L) is added directly based on a certain rule, e.g. add 6 g/L glucose if the measured glucose concentration drops below 5 g/L glucose in the cell culture.

By varying the addition rate in the continuous processes (e.g. from 0.5 to 0.7 g/h) or by altering the amount of glucose in a bolus addition depending e.g. on a measured concentration of glucose in the culture medium (e.g. adding 4 g/L glucose when the glucose level is below 4 g/L, or adding 6 g/L when the glucose level is below 4 g/L), the amount of glucose that is added into the cell culture over the process to achieve the desired average over the production phase can be varied.

The results are demonstrated in FIG. 2. There is a strong correlation between the glucose average concentration from day −7 to day 0 and the relative Fab high mannose level.

Example 1 demonstrates that, at any scale, increasing the average glucose concentration in the culture medium over day −7 to day 0 of the fermentation results in an increase in the percentage of the high mannose glycoform of the antibody/Fab region expressed by the cells relative to the total amount of glycosylated antibody/Fab region.

### Example 2

Regulation of Fab Glycosylation by Varying the Glucose Addition

To confirm the results of Example 1 in a dedicated experiment, an experiment in 1 L bioreactors was conducted using the methods of the disclosure. The cultivation was performed using a CHO-K1 cell line producing an antibody with Fc and Fab glycosylation. The fermentation was conducted with a fed batch standard process for Gantenerumab and using a chemically-defined standard medium, containing glucose. The harvest was performed on day 0 and the glycoform distribution was analysed from centrifuged and Protein A purified samples.

The only variation was the glucose addition strategy. Apart from minor variations that can be ignored according to process experience (e.g. agitation, aeration, cell bank, cell age), the parameters are the same for all runs apart from the glucose addition.

From day −10 of the production phase the glucose concentration was measured daily using the Cedex Bio HT. After the measurement a 500 g/L glucose solution was added to each bioreactor every day, with a variation in the concentration added:
glucose solution is added to a daily concentration of 7 g/L;
glucose solution is added to a daily concentration of 9 g/L;
glucose solution is added to a daily concentration of 12 g/L; or
glucose solution is added to a daily concentration of 15 g/L.

The calculation for the glucose additions was based on the daily measurement.

There is a strong correlation between the average glucose concentration from day −7 to day 0 in the culture over all or a part of the production phase (calculated with the formulae herein) and with the relative content of the Fab high mannose glycoform of the expressed antibody.

The results are presented in FIG. 3A which clearly shows the correlation between the average glucose concentration from day −7 to day 0 of the production phase and the production of Fab high mannose glycoforms of the monoclonal antibody. In FIG. 3B the Fab high mannose sum is separated into its parts of mannose 5, 6 and 7. All separate parts of the sum of Fab high mannose show the same trend and dependency on the glucose average level in the reactor. In FIG. 3C the calculation of the average glucose level from day −7 to day 0 is explained. The calculation is based on the daily measured glucose concentration in the culture from samples taken before the glucose addition and the calculated glucose concentration in the culture after the bolus addition addition (calculated from the measured value before the addition and the added amount of glucose).

Results (see also FIG. 3A):
Glucose addition to 7 g/L→Glucose average day −7 to day 0 is 5.7 g/L→about 9% Fab high man
Glucose addition to 9 g/L→Glucose average day −7 to day 0 is 7.4 g/L→about 10.5% Fab high man
Glucose addition to 12 g/L→Glucose average day −7 to day 0 is 10.6 g/L→about 13% Fab high man
Glucose addition to 15 g/L→Glucose average day −7 to day 0 is 12.4 g/L→about 15% Fab high man

Example 2 shows the effect of different glucose addition regimes on the production of high mannose glycoforms of the antibody, with an increase in the daily glucose addition and thus an increase in the glucose average level from day −7 to day 0, resulting in an increase in the percentage of Fab high mannose glycoforms, particularly in the amount of Man5 and Man6 and to a lesser extent in the amount of Man7, in the glycan.

### Example 3

Pharmacokinetics of Gantenerumab

Clinical Study: Subcutaneous Gantenerumab Injection, Study 1

Gantenerumab was produced according to the methods of the invention (referred to hereinafter as G4 process). The percentage of each Fab glycoform resulting from the G4 process is as indicated in FIG. 6A/6B and can be compared to the Fab glycoform resulting from a G3 process. As set out above, a G3 process is a previous process for producing a high mannose content (e.g. more than 8% high mannose glycoforms), in the same antibody and is not the process described herein. The high mannose glycoforms resulting from the G3 process thus act as a reference product.

The pharmacokinetics of the Gantenerumab glycoforms resulting from the G4 process and the G3 process were compared in a clinical study. The study was multi-centre, randomized, open-label, single-dose, parallel group study in healthy volunteers.

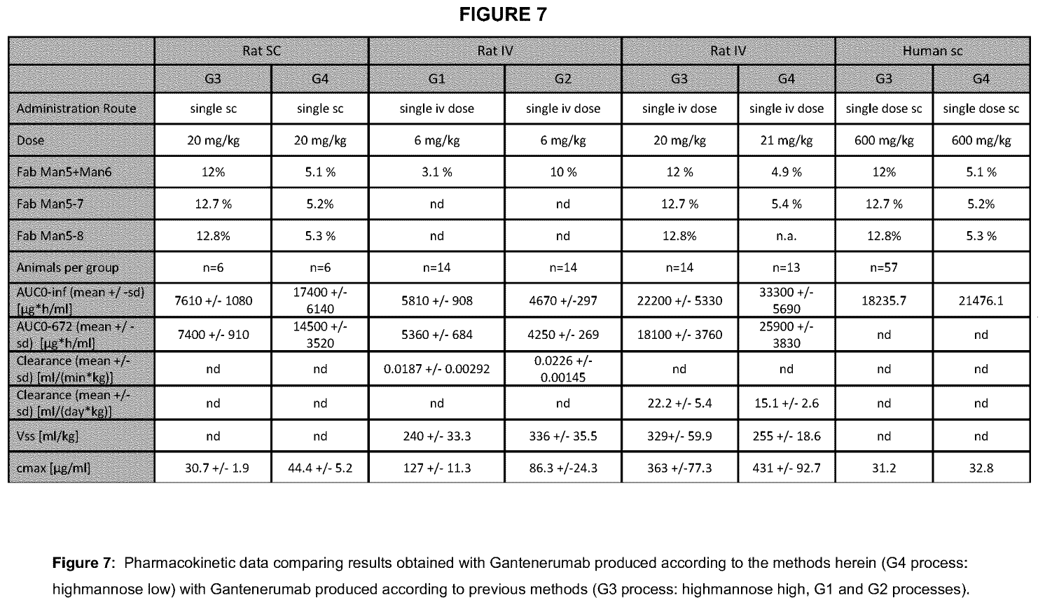

Following s.c. administration, Gantenerumab was absorbed slowly with peak plasma concentrations reached at a median time of 95.5 hours and 110 hours for material produced by the G3 and G4 processes, respectively. The plasma exposure in terms of AUC 0-inf was approximately 1.18 fold higher after sc administration of 600 mg Gantenerumab produced by the G4 process compared to 600 mg Gantenerumab produced by the G3 process, whereas the Cmax results were similar (5.1% higher after administration of Gantenerumab produced by the G4 process) (see also FIG. 7). Pharmacokinetic parameters were derived according to standard non-compartmental analysis (NCA) methods using WinNonlin version 6.3 (Pharsight, Mountain View, CA, USA). The statistical analysis was performed with a linear model with the PK parameter (log scale) as dependent variable and the independent fixed factors of “treatment”, “study center”, “sex”, and “body weight category” at randomisation.

FIGS. 4A and 4B further show that the main difference between the product of the G3 process and the glycoforms produced according to the invention (i.e. a G4 process) occurs in the first 288 hours after administration of Gantenerumab. An increase in bioavailability of about 18% can be achieved using the product of the G4 process prepared according to the methods herein, which comprises 5.2% Man5-7, over the product of the G3 process which comprises 12.7% Man5-7 (see FIG. 7).

Rat, Subcutaneous Gantenerumab Injection, Study 2

12 rats (n=6 per group) received material produced by the G4 process (5.2% Mannose 5-7) and material produced by the G3 process (12.7% Man5-Man7), respectively, in a single dose subcutaneously (neck region) at a nominal dose of 20 mg/kg to two parallel groups of male Wistar rats. Serial blood samples were collected over 4 weeks from each animal. The concentration of Gantenerumab in Wistar rats K3-EDTA plasma samples was analysed by a qualified ECLIA methods specific for human Ig/FabCH1/kappa domain using a Cobas e411 instrument. Briefly, test samples of Gantenerumab, first detection antibody mAbHFab(kappa)M-1.7.10-IgG-Bi, second detection antibody mAbHFabCH1M1.19.31-IgGRu, and SA-beads are added stepwise to a detection vessel and incubated for 9 minutes in each step. Finally, the SA-bead-bound complex is detected by a measuring cell which numbers the counts of SA-beads in repeat. The counts are proportional to the analyte concentration in the test sample.

Pharmacokinetic assessment was performed by non-compartmental analysis. Average dose-normalized AUC (0-last) for G4 process-produced material was higher and accounted for about 228% of that of G3 process-produced material (FIG. 7). Moreover, Cmax was 44% higher in the highmannose low material compared to the highmannose high material. These data further demonstrate that the G4 process-produced Gantenerumab stays longer in the circulation, i.e. has reduced clearance and has a better bioavailability than the G3 process-produced Gantenerumab.

Rat, Intravenous Gantenerumab Injection Study 3

Further, plasma concentrations of total Gantenerumab and Gantenerumab with Man5/Man6 Fab glycans were determined following intravenous administration of Gantenerumab to rats (15 mg/kg). Gantenerumab concentrations were analyzed by ELISA. In addition, for glycan determination Gantenerumab was extracted from plasma by immunoaffinity purification at various times after dosing. Glycan composition of extracted Gantenerumab was determined by an LC-MS method. The obtained sum of Gantenerumab Man5/Man6 glycans (FHMG) was used to estimate the fraction of Gantenerumab containing at least one Man5/Man6 glycan (HMGant) assuming a statistical distribution of the high mannose glycans according to HMGant=2×(FHMG−(FHMG×FHMG))+(FHMG×FHMG). Concentrations of Gantenerumab with at least one Man5/Man6 glycan were calculated by multiplying total Gantenerumab concentration from ELISA by HMGant.

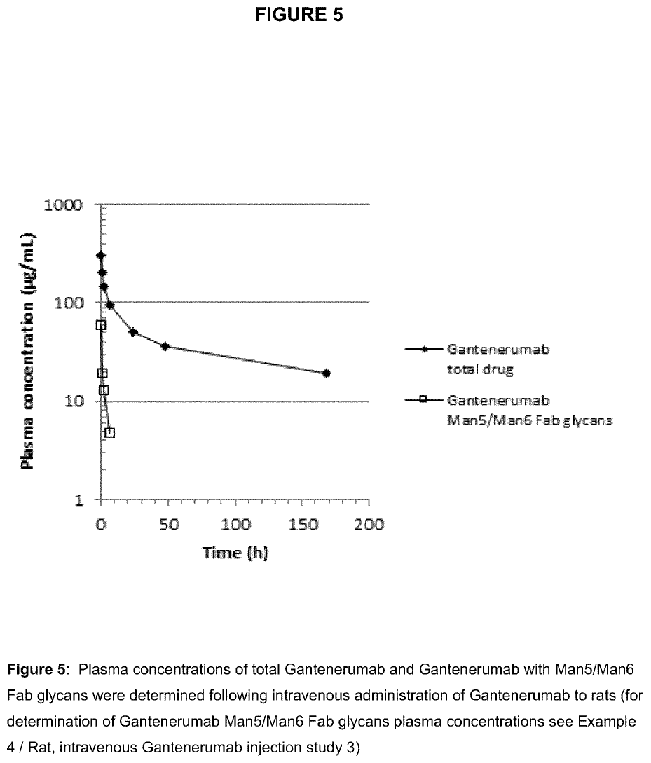

The results, indicated in FIG. 5, demonstrate a rapid loss of Gantenerumab Man5/Man6 glycans from circulation, so that they were no longer detectable by 24 hours post dose.

Rat: Intravenous Gantenerumab Injection, Study 4

28 rats (n=13-14 per group) received G4 process-produced material (5.4% Mannose 5-7) and G3 process-produced material (12.7% Man5-Man7), respectively in a single dose intravenously at a nominal dose of 20 mg/kg to two parallel groups of male Wistar rats. Serial blood samples were collected over 4 weeks from each animal. The concentration of Gantenerumab in Wistar rats K3-EDTA plasma samples were analysed by a qualified ECLIA method specific for human Ig/FabCH1/kappa domain using a Cobas e411 instrument. Briefly, test samples of Gantenerumab, first detection antibody mAbHFab(kappa)M-1.7.10-IgG-Bi, second detection antibody mAbHFabCH1M1.19.31-IgGRu, and SA-beads are added stepwise to a detection vessel and incubated for 9 minutes in each step. Finally, the SA-bead-bound complex is detected by a measuring cell which numbers the counts of SA-beads in repeat. The counts are proportional to the analyte concentration in the test sample.

Pharmacokinetic assessment was performed by non-compartmental analysis. Average dose-normalized AUC (0-last) for G4 process-produced material was higher and accounted for about 136% of that of G3 process-produced material (FIG. 7) and the average clearance for G4 process-produced material (15.1 ml/day/kg) accounted for 68% of that of G3 process-produced material (22.2 ml/day/kg). Average apparent volumes of distribution (Vss) were 329 and 255 ml/kg for G3 process-produced and G4 process-produced material, respectively (FIG. 7). These data demonstrate that the highmannose low Gantenerumab stays longer in the circulation and has a better bioavailability than the highmannose high Gantenerumab.

### Example 4

Rat: Intravenous Gantenerumab Injection, Study 5

Material produced by G1 and G2 processes represents Gantenerumab produced by previous production processes (which processes are different from the methods of the present invention and different from the G3 method). Man5+Man6 content is less than 8%, typically about 3.1% in the G1 process-produced material, whereas it is more than 8%, typically about 10% in the G2 process-produced material. The glycan composition was analyzed by LC-MS after digestion of extracted Gantenerumab from plasma samples after immunoprecipitation. 28 rats received material produced by G1 or G2 processes (n=14 per group), in a single dose intravenously at a nominal dose of 6 mg/kg to two parallel groups of male Wistar rats. Serial blood samples were collected over 4 weeks from each animal. The concentration of Gantenerumab in Wistar rats plasma samples were analysed by ELISA. Pharmacokinetic assessment was performed by non-compartmental methods.

AUC 0-inf for the G2 process-produced material was lower and accounted for about 80% of that of the G1 process-produced material. Cmax was also lower (see also FIG. 7), demonstrating a better bioavailability when the Man5+Man6 content is lower in the antibody.

Effect of Fc/Fab Glycosylation on Gantenerumab Clearance

A single dose PK study was performed in rats. Parallel groups of rat were administered a single dose of Gantenerumab (G2 process-produced). Samples were collected up to 24 hours or 48 hours after dosing. Gantenerumab was extracted from plasma by immunoprecipitation and the glycan composition was analysed by LC-MS after digestion of the extract.

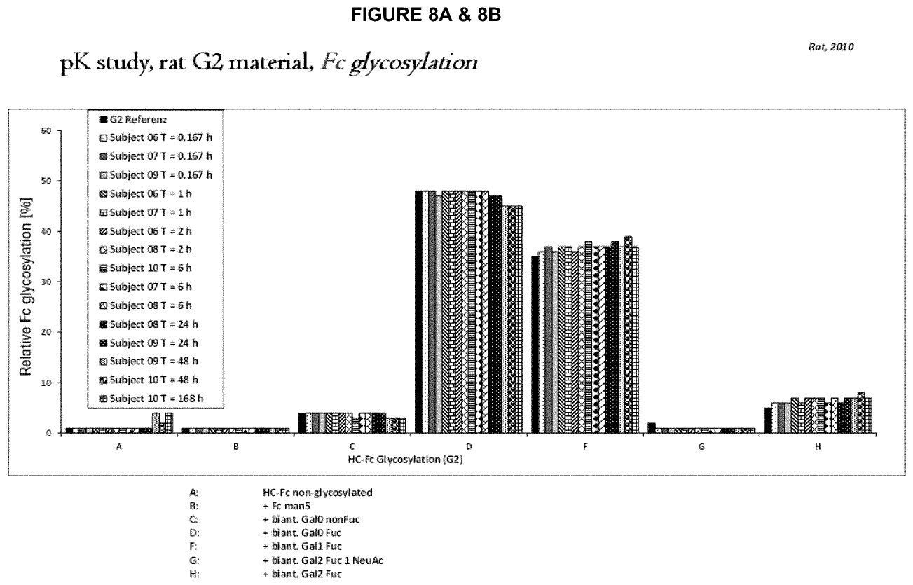

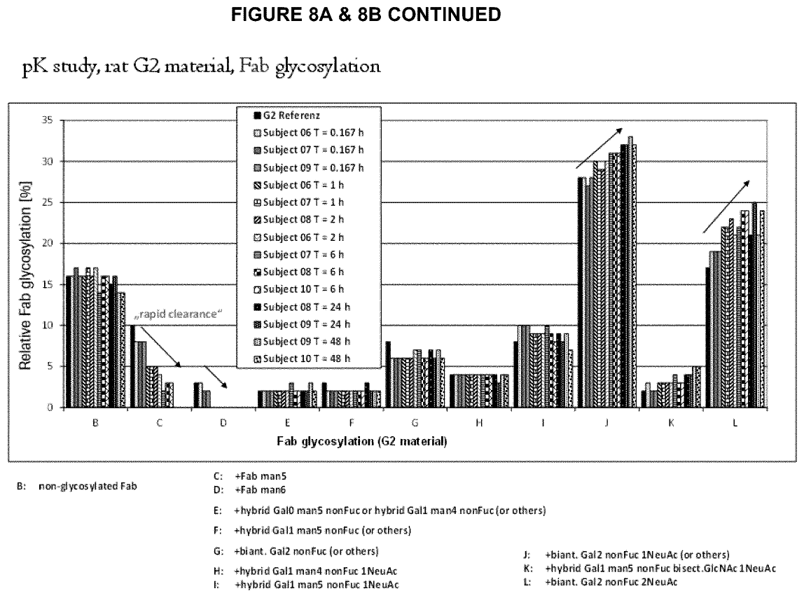

FIG. 8B shows the percentage of a specific glycoform of Gantenerumab (produced by a G2 process) measurable after up to 48 hours post-dose, demonstrating a very rapid clearance of the Man5 and Man6 Fab glycoforms. In contrast, as can be seen from FIG. 8A, the Fc glycostructure in the G2 process-produced material does not have an influence on the clearance of individual glycoforms.

The following sequences are referred to in this disclosure:

SEQ ID NO: 1 = Gantenerumab VH CDR1
Gly Phe Thr Phe Ser Ser Tyr Ala Met Ser
SEQ ID NO: 2 = Gantenerumab VH CDR2
Ala Ile Asn Ala Ser Gly Thr Arg Thr Tyr Tyr Ala Asp Ser Val Lys Gly
SEQ ID NO: 3 = Gantenerumab VH CDR3
Gly Lys Gly Asn Thr His Lys Pro Tyr Gly Tyr Val Arg Tyr Phe Asp Val
SEQ ID NO: 4 = Gantenerumab VL CDR1
Arg Ala Ser Gln Ser Val Ser Ser Ser Tyr Leu Ala
SEQ ID NO: 5 = Gantenerumab VL CDR2
Gly Ala Ser Ser Arg Ala Thr
SEQ ID NO: 6 = Gantenerumab VL CDR3
Leu Gln Ile Tyr Asn Met Pro Ile
SEQ ID NO: 7 = Gantenerumab V_{H} domain
Gln Val Glu Leu Val Glu Ser Gly Gly Gly Leu Val Gln Pro Gly Gly Ser Leu Arg Leu Ser Cys Ala Ala Ser
Gly Phe Thr Phe Ser Ser Tyr Ala Met Ser Trp Val Arg Gln Ala Pro Gly Lys Gly Leu Glu Trp Val Ser Ala
Ile Asn Ala Ser Gly Thr Arg Thr Tyr Tyr Ala Asp Ser Val Lys Gly Arg Phe Thr Ile Ser Arg Asp Asn Ser
Lys Asn Thr Leu Tyr Leu Gln Met Asn Ser Leu Arg Ala Glu Asp Thr Ala Val Tyr Tyr Cys Ala Arg Gly
Lys Gly Asn Thr His Lys Pro Tyr Gly Tyr Val Arg Tyr Phe Asp Val Trp Gly Gln Gly Thr Leu Val Thr Val
Ser Ser
SEQ ID NO: 8 = Gantenerumab V_{L} domain
Asp Ile Val Leu Thr Gln Ser Pro Ala Thr Leu Ser Leu Ser Pro Gly Glu Arg Ala Thr Leu Ser Cys Arg Ala
Ser Gln Ser Val Ser Ser Ser Tyr Leu Ala Trp Tyr Gln Gln Lys Pro Gly Gln Ala Pro Arg Leu Leu Ile Tyr
Gly Ala Ser Ser Arg Ala Thr Gly Val Pro Ala Arg Phe Ser Gly Ser Gly Ser Gly Thr Asp Phe Thr Leu Thr
Ile Ser Ser Leu Glu Pro Glu Asp Phe Ala Thr Tyr Tyr Cys Leu Gln Ile Tyr Asn Met Pro Ile Thr Phe Gly
Gln Gly Thr Lys Val Glu Ile Lys Arg Thr
SEQ ID NO: 9 = Gantenerumab Heavy Chain
Gln Val Glu Leu Val Glu Ser Gly Gly Gly Leu Val Gln Pro Gly Gly Ser Leu Arg Leu Ser Cys Ala Ala Ser
Gly Phe Thr Phe Ser Ser Tyr Ala Met Ser Trp Val Arg Gln Ala Pro Gly Lys Gly Leu Glu Trp Val Ser Ala
Ile Asn Ala Ser Gly Thr Arg Thr Tyr Tyr Ala Asp Ser Val Lys Gly Arg Phe Thr Ile Ser Arg Asp Asn Ser
Lys Asn Thr Leu Tyr Leu Gln Met Asn Ser Leu Arg Ala Glu Asp Thr Ala Val Tyr Tyr Cys Ala Arg Gly
Lys Gly Asn Thr His Lys Pro Tyr Gly Tyr Val Arg Tyr Phe Asp Val Trp Gly Gln Gly Thr Leu Val Thr Val
Ser Ser Ala Ser Thr Lys Gly Pro Ser Val Phe Pro Leu Ala Pro Ser Ser Lys Ser Thr Ser Gly Gly Thr Ala
Ala Leu Gly Cys Leu Val Lys Asp Tyr Phe Pro Glu Pro Val Thr Val Ser Trp Asn Ser Gly Ala Leu Thr Ser
Gly Val His Thr Phe Pro Ala Val Leu Gln Ser Ser Gly Leu Tyr Ser Leu Ser Ser Val Val Thr Val Pro Ser
Ser Ser Leu Gly Thr Gln Thr Tyr Ile Cys Asn Val Asn His Lys Pro Ser Asn Thr Lys Val Asp Lys Lys Val
Glu Pro Lys Ser Cys Asp Lys Thr His Thr Cys Pro Pro Cys Pro Ala Pro Glu Leu Leu Gly Gly Pro Ser Val
Phe Leu Phe Pro Pro Lys Pro Lys Asp Thr Leu Met Ile Ser Arg Thr Pro Glu Val Thr Cys Val Val Val Asp
Val Ser His Glu Asp Pro Glu Val Lys Phe Asn Trp Tyr Val Asp Gly Val Glu Val His Asn Ala Lys Thr Lys
Pro Arg Glu Glu Gln Tyr Asn Ser Thr Tyr Arg Val Val Ser Val Leu Thr Val Leu His Gln Asp Trp Leu Asn
Gly Lys Glu Tyr Lys Cys Lys Val Ser Asn Lys Ala Leu Pro Ala Pro Ile Glu Lys Thr Ile Ser Lys Ala Lys
Gly Gln Pro Arg Glu Pro Gln Val Tyr Thr Leu Pro Pro Ser Arg Asp Glu Leu Thr Lys Asn Gln Val Ser Leu
Thr Cys Leu Val Lys Gly Phe Tyr Pro Ser Asp Ile Ala Val Glu Trp Glu Ser Asn Gly Gln Pro Glu Asn Asn
Tyr Lys Thr Thr Pro Pro Val Leu Asp Ser Asp Gly Ser Phe Phe Leu Tyr Ser Lys Leu Thr Val Asp Lys Ser
Arg Trp Gln Gln Gly Asn Val Phe Ser Cys Ser Val Met His Glu Ala Leu His Asn His Tyr Thr Gln Lys Ser
Leu Ser Leu Ser Pro Gly Lys
SEQ ID NO: 10 = Gantenerumab Light Chain
Asp Ile Val Leu Thr Gln Ser Pro Ala Thr Leu Ser Leu Ser Pro Gly Glu Arg Ala Thr Leu Ser Cys Arg Ala
Ser Gln Ser Val Ser Ser Ser Tyr Leu Ala Trp Tyr Gln Gln Lys Pro Gly Gln Ala Pro Arg Leu Leu Ile Tyr
Gly Ala Ser Ser Arg Ala Thr Gly Val Pro Ala Arg Phe Ser Gly Ser Gly Ser Gly Thr Asp Phe Thr Leu Thr
Ile Ser Ser Leu Glu Pro Glu Asp Phe Ala Thr Tyr Tyr Cys Leu Gln Ile Tyr Asn Met Pro Ile Thr Phe Gly
Gln Gly Thr Lys Val Glu Ile Lys Arg Thr Val Ala Ala Pro Ser Val Phe Ile Phe Pro Pro Ser Asp Glu Gln
Leu Lys Ser Gly Thr Ala Ser Val Val Cys Leu Leu Asn Asn Phe Tyr Pro Arg Glu Ala Lys Val Gln Trp
Lys Val Asp Asn Ala Leu Gln Ser Gly Asn Ser Gln Glu Ser Val Thr Glu Gln Asp Ser Lys Asp Ser Thr Tyr
Ser Leu Ser Ser Thr Leu Thr Leu Ser Lys Ala Asp Tyr Glu Lys His Lys Val Tyr Ala Cys Glu Val Thr His
Gln Gly Leu Ser Ser Pro Val Thr Lys Ser Phe Asn Arg Gly Glu Cys
SEQ ID NO: 11 = heavy chain Fab fragment for antibody binding to human transferrin receptor
Gln Ser Met Gln Glu Ser Gly Pro Gly Leu Val Lys Pro Ser Gln Thr Leu Ser Leu Thr Cys Thr Val Ser
Gly Phe Ser Leu Ser Ser Tyr Ala Met Ser Trp Ile Arg Gln His Pro Gly Lys Gly Leu Glu Trp Ile Gly Tyr
lle Trp Ser Gly Gly Ser Thr Asp Tyr Ala Ser Trp Ala Lys Ser Arg Val Thr Ile Ser Lys Thr Ser Thr Thr
Val Ser Leu Lys Leu Ser Ser Val Thr Ala Ala Asp Thr Ala Val Tyr Tyr Cys Ala Arg Arg Tyr Gly Thr
Ser Tyr Pro Asp Tyr Gly Asp Ala Ser Gly Phe Asp Pro Trp Gly Gln Gly Thr Leu Val Thr Val Ser Ser
Ala Ser Val Ala Ala Pro Ser Val Phe Ile Phe Pro Pro Ser Asp Glu Gln Leu Lys Ser Gly Thr Ala Ser Val
Val Ccy Leu Leu Asn Asn Phe Tyr Pro Arg Glu Ala Lys Val Gln Trp Lys Val Asp Asn Ala Leu Gln Ser
Gly Asn Ser Gln Glu Ser Val Thr Glu Gln Asp Ser Lys Asp Ser Thr Tyr Ser Leu Ser Ser Thr Leu Thr
Leu Ser Lys Ala Asp Tyr Glu Lys His Lys Val Tyr Ala Cys Glu Val Thr His Gln Gly Leu Ser Ser Pro
Val Thr Lys Ser Phe Asn Arg Gly Glu Cys
SEQ ID NO: 12 = light chain for antibody binding to human transferrin receptor
Ala Ile Gln Leu Thr Gln Ser Pro Ser Ser Leu Ser Ala Ser Val Gly Asp Arg Val Thr Ile Thr Cys Arg Ala
Ser Gly Ser Ile Ser Ser Tyr Leu Ala Trp Tyr Gln Gln Lys Pro Gly Lys Ala Pro Lys Leu Leu Ile Tyr Arg
Ala Ser Thr Leu Ala Ser Gly Val Pro Ser Arg Phe Ser Gly Ser Gly Ser Gly Thr Asp Phe Thr Leu Thr Ile
Ser Ser Leu Gln Pro Glu Asp Phe Ala Thr Tyr Tyr Cys Gln Gln Asn Tyr Ala Ser Ser Asn Val Asp Asn
Thr Phe Gly Gly Gly Thr Lys Val Glu Ile Lys Ser Ser Ala Ser Thr Lys Gly Pro Ser Val Phe Pro Leu Ala
Pro Ser Ser Lys Ser Thr Ser Gly Gly Thr Ala Ala Leu Gly Cys Leu Val Lys Asp Tyr Phe Pro Glu Pro Val
Thr Val Ser Trp Asn Ser Gly Ala Leu Thr Ser Gly Val His Thr Phe Pro Ala Val Leu Gln Ser Ser Gly Leu
Tyr Ser Leu Ser Ser Val Val Thr Val Pro Ser Ser Ser Leu Gly Thr Gln Thr Tyr Ile Cys Asn Val Asn His
Lys Pro Ser Asn Thr Lys Val Asp Lys Lys Val Glu Pro Lys Ser Cys

1. A composition comprising a glycosylated monoclonal antibody, wherein said antibody is an anti-human Abeta antibody, a bi-specific antibody comprising an anti-human Abeta antibody, or a fragment of an anti-human Abeta antibody comprising a glycosylated Fab region and capable of binding Abeta, the antibody having N-glycosylation in the Fab region(s) thereof, wherein relative to the total amount of glycosylated Fab in the composition, about 20% or less of Fab regions in the composition have an N-linked high mannose glycan.

2. The composition of claim 1 which is a pharmaceutical composition or a cell culture supernatant obtainable during or after recombinant production of the antibody.

3. A method for reducing the rate at which a glycosylated monoclonal antibody, wherein said antibody is an anti-human Abeta antibody, a bi-specific antibody comprising an anti-human Abeta antibody, or a fragment of an anti-human Abeta antibody comprising a glycosylated Fab region and capable of binding Abeta, clears from the circulation of an animal to which the antibody has been administered, which method comprises regulating the relative content of high mannose Fab glycoforms of the glycosylated monoclonal antibody in a composition comprising the antibody.

4. The method of claim 3 wherein regulating the relative content of high mannose Fab glycoforms of the glycosylated monoclonal antibody in a composition comprising the antibody comprises maintaining an average concentration of glucose in a culture medium used for producing the glycosylated monoclonal antibody by fermentation therein of a eukaryotic cell expressing the monoclonal antibody, during all or a part of the production phase of the fermentation.

5. A method for regulating the relative content of high mannose Fab glycoforms of a glycosylated monoclonal antibody in a composition, wherein said antibody is an anti-human Abeta antibody, a bi-specific antibody comprising an anti-human Abeta antibody, or a fragment of an anti-human Abeta antibody comprising a glycosylated Fab region and capable of binding Abeta, the method comprising, during the production phase of fermentation, optimising the concentration of a carbohydrate source for a eukaryotic cell in a culture medium used for producing the glycosylated monoclonal antibody by fermentation therein of the eukaryotic cell expressing the monoclonal antibody.

6. The method of claim 5 comprising maintaining an average concentration of glucose in the culture medium during all or a part of the production phase.

7. The method of claim 5 or claim 6 further comprising a step of recovering the antibody from the culture medium.

8. The method of any one of claims 3 to 7 wherein the high mannose Fab glycoforms make up about 20% or less of the total Fab glycoforms of the monoclonal antibody.

9. The composition of claim 1 or 2 or method of any one of claims 3 to 8 wherein the percentage of Fab regions having N-linked high mannose glycan is from about 0-20%, about 0-15%, about 0-12% or about 0-10%, optionally wherein the percentage of Fab regions having N-linked high mannose glycan is from about 2-20%, about 2-15%, about 2-12% or about 2-10% or about 4-10%, about 4-12%, about 4-15% or about 4-20%.

10. The composition or method of claim 9 wherein the high mannose glycan is one or a mixture of Man5, Man6 and Man7 glycoforms, optionally wherein the mannose residues in the high mannose glycan are Man5, Man 6 and Man7.

11. The composition or method of any one of claims 1 to 10 wherein the monoclonal antibody is Gantenerumab and comprises:
(a) a V_{H} and a V_{L} CDR amino acid sequences as set out in SEQ ID Nos:1-6, above, a V_{H} and a V_{L} domain amino acid sequences of SEQ ID NO:7 and SEQ ID NO:8, or a heavy and light chain comprising the amino acid sequence of SEQ ID NO:9 and 10;
(b) a V_{H} CDR1 comprising the amino acid sequence of SEQ ID NO:1; a V_{H} CDR2 comprising the amino acid sequence of SEQ ID NO:2; a V_{H} CDR3 comprising the amino acid sequence of SEQ ID NO:3; a V_{L} CDR1 comprising the amino acid sequence of SEQ ID NO:4; a V_{L} CDR2 comprising the amino acid sequence of SEQ ID NO:5; and a V_{L} CDR3 comprising the amino acid sequence of SEQ ID NO:6;
(c) a V_{H} domain comprising the amino acid sequence of SEQ ID NO:7; and a V_{L} domain comprising the amino acid sequence of SEQ ID NO:8; or
(d) a heavy chain comprising the amino acid sequence of SEQ ID NO:9; and a light chain comprising the amino acid sequence of SEQ ID NO:10;
said composition comprising about 20% or less of a high mannose glycoform of said antibody relative to the total amount of V_{H} glycosylated antibody in the composition, wherein said glycosylation is N-glycosylation at Asn52 in SEQ ID NO:9.

12. The composition or method of claim 11 wherein, when the monoclonal antibody is a bi-specific antibody, one of the binding specificities is for human A-beta and the other specificity is for the transferrin receptor.

13. The composition of method of claim 12, wherein the monoclonal antibody is a bi-specific antibody comprising:
a heavy chain with the amino acid sequence of SEQ ID NO:9;
a light chain with the amino acid sequence of SEQ ID NO:10;
a heavy chain Fab fragment with an amino acid sequence of SEQ ID NO:11; and
a light chain with an amino acid sequence of SEQ ID NO:12.

14. The method of any one of claims 3 to 13, wherein the average glucose concentration in the culture medium over the production phase in the recombinant production of the monoclonal antibody is from about 0.5 g/L to about 18 g/L.

15. The method of claim 14, wherein the concentration of glucose is averaged over day −7 to day 0 of the production phase.

16. The method of any one of claims 3 to 15 wherein the concentration of glucose is monitored and controlled in the culture medium to achieve an average concentration thereof over all or a part of the production phase, optionally over day −7 to day 0 of the production phase.

17. The method of any one of claims 3 to 16 wherein:
(a) the desired relative content of high mannose Fab glycoforms of the glycosylated monoclonal antibody resulting from the fermentation is about 3%, the average concentration of glucose in the culture medium between about day −7 and day 0 is between about 0 g/L and about 3 g/L;
(b) the desired relative content of high mannose Fab glycoforms of the glycosylated monoclonal antibody resulting from the fermentation is about 7%, the average concentration of glucose in the culture medium between about day −7 and day 0 is between about 3 g/L and about 6 g/L;
(b) the desired relative content of high mannose Fab glycoforms of the glycosylated monoclonal antibody resulting from the fermentation is about 10.5%, the average concentration of glucose in the culture medium between about day −7 and day 0 is between about 6 g/L and about 9 g/L;
(c) the desired relative content of high mannose Fab glycoforms of the glycosylated monoclonal antibody resulting from the fermentation is about 13%, the average concentration of glucose in the culture medium between about day −7 and day 0 is between about 9 g/L and about 11 g/L; or
(d) the desired relative content of high mannose Fab glycoforms of the glycosylated monoclonal antibody resulting from the fermentation is about 15%, the average concentration of glucose in the culture medium between about day −7 and day 0 is between about 11 g/L and about 14 g/L.

18. A monoclonal antibody composition obtainable by the method of any one of claims 3 to 17.

19. A composition according to any one of claims 1 to 13, for use in the diagnosis or treatment of a disease in an individual suspected of or suffering therefrom, optionally wherein the disease is dementia, Alzheimer's Disease, motor neuropathy, Parkinson's Disease, amylotrophic lateral sclerosis (ALS), scrapie, HIV-related dementia, Creutzfeld-Jakob disease (CJD), hereditary cerebral haemorrhage, Down's syndrome and neuronal disorders related to ageing; and cancer, such as metastatic colorectal cancer, metastatic non-small cell lung cancer, ovarian cancer and head and neck cancer.

20. The composition or method according to any one of claims 1 to 19 wherein the relative amount of an N-linked high mannose glycan in the Fab region(s) to the total amount of glycosylated Fab in the composition is analysed by hydrophilic-interaction chromatography-ultra high performance liquid chromatography (HILIC-UHPLC) with subsequent fluorescence detection of 2-aminobenzamide labelled glycans.
